(19) United States
(12) Patent Application Publication
(10) Pub. No.: US 2026/0049933 A1
(43) Pub. Date: Feb. 19, 2026

(54) ANALYZER FOR AUTOMATICALLY DETECTING AND QUANTIFYING GLUTARALDEHYDE IN BIOCIDE PRODUCTS

(71) Applicant: Saudi Arabian Oil Company, Dhahran (SA)

(72) Inventors: Xiangyang Zhu, Dhahran (SA); Mazen A. Al-Saleh, Al Khobar (SA); Fahad N. Al-Abeedi, Dammam (SA); Alexander Grigoryan, Dhahran (SA); Juan F. Hernandez Montiel, Al Khobar (SA)

(73) Assignee: Saudi Arabian Oil Company, Dhahran (SA)

(21) Appl. No.: 18/807,341

(22) Filed: Aug. 16, 2024

(51) Int. Cl. G01N 21/64; B01L 3/00; G01N 21/85; G01N 33/18; G16C 20/30

(52) U.S. Cl. CPC G01N 21/6428; B01L 3/502; G01N 21/85; G01N 33/1826; G16C 20/30; B01L 2200/0684; B01L 2200/16; B01L 2300/047; B01L 2300/0663; B01L 2300/0681; B01L 2300/0877; B01L 2300/1805; B01L 2400/0487; B01L 2400/0622; G01N 2021/6421; G01N 2021/8571; G01N 2333/765

(57) ABSTRACT

An analyzer is disclosed for detecting and quantifying a glutaraldehyde (GLUT) composition in a water sample. The analyzer includes a peristaltic pump and a three-way valve, where the pump draws in a water sample comprising a GLUT composition through the 3-way valve. The analyzer also includes a 6-way valve that receives the water sample and a bovine serum albumin-coated gold (Au-BSA) nanoclusters reagent. The analyzer also includes: a debubbler that receive a mixture of the water sample and the reagent and removes bubbles from the mixture; and a reaction coil that receives the water sample and the reagent from the debubbler, such that they are reacted. The analyzer further includes a fluorescence flow cell that receives the reacted mixture; measures a fluorescence intensity of the Au-BSA nanoclusters in the mixture; and determines a concentration of the GLUT composition in the water sample based on the fluorescence intensity.

### TECHNICAL FIELD

The present disclosure is generally related to a device for detecting and quantifying glutaraldehyde in water.

### BACKGROUND

Glutaraldehyde (GLUT) is a primary component in biocide products commonly used in the oil and gas industry for microbial control in water systems. Biocidal performance generally depends on the concentration and contact time with microorganisms, and thus it is important to monitor biocide residual concentration closely in any water system to ensure microbial control.

GLUT-based biocides are readily degradable and thus are generally considered environmentally friendly. Conventionally, GLUT is detected and quantified using colorimetric methods using commercial test kits, such as Glutaraldehyde Test Kit GT-1 (Cat No. 2587200). However, in a large water pipeline networks, it is extremely challenging to collect water samples for biocide residual measurement at downstream locations of the pipeline network, especially in remote locations, after a batch treatment of the biocide product at an upstream location. This is due to the difficulties to estimate the biocide travel time in the large pipeline network because of the complexity of pipeline network (pressure, diameters, branches, etc.), working schedule, and daily operation changes and fluctuations (e.g., flow rate).

Accordingly, there is a need for effective field measurement tools for GLUT-based biocides in water networks, particularly those in oil and gas facilities. The present application addresses these and other challenges.

### SUMMARY

In a first aspect, an analyzer for detecting and quantifying a glutaraldehyde (GLUT) composition in a water sample is provided. The analyzer includes a peristaltic pump and a three-way valve operatively connected to the peristaltic pump, wherein the peristaltic pump is configured to draw in a water sample comprising a GLUT composition through the 3-way valve. The analyzer also includes a six-way valve operatively connected to the three-way valve via the peristaltic pump, wherein the six-way valve is configured to receive: a) the water sample from the 3-way valve via a first inlet and b) a bovine serum albumin-coated gold (Au-BSA) nanoclusters reagent via a second inlet. The analyzer also includes a debubbler operatively connected to the six-way valve and configured to receive a mixture of the water sample and the Au-BSA nanoclusters reagent and remove bubbles from the mixture. The analyzer further includes a reaction coil operatively connected to the debubbler and configured to receive the water sample and the Au-BSA nanoclusters reagent, wherein the water sample and the Au-BSA nanoclusters reagent are mixed and reacted in the reaction coil. The analyzer also includes a fluorescence flow cell comprising a 375 nm light-emitting diode (LED), wherein the flow cell is configured to: receive the mixture from the reaction coil; measure a fluorescence intensity of the Au-BSA nanoclusters in the mixture at an emission wavelength of 675 nm, wherein the Au-BSA nanoclusters comprises a maximum excitation wavelength of approximately 375 nm and a maximum emission wavelength of approximately 675 nm; and determine a presence and concentration of the GLUT composition in the water sample based on a comparison between the measured fluorescence intensity of the Au-BSA nanoclusters at the emission wavelength of 675 nm with fluorescence intensity values of calibration samples comprising Au-BSA nanoclusters and known glutaraldehyde concentrations.

In another aspect, a correlation of the fluorescence intensity of the Au-BSA nanoclusters and the concentration of the GLUT composition is based on the equation: Y=a+b\sqrt{X}, wherein Y is the peak height of the fluorescence signal, X is the concentration of GLUT composition in the water sample, and a and b are calibration factors determined using a 2-point calibration procedure.

In a further aspect, to perform the 2-point calibration procedure the fluorescence flow cell is configured to: measure a fluorescence intensity of the Au-BSA nanoclusters at an emission wavelength of 675 nm in a mixture of a water sample without the GLUT composition and the Au-BSA nanoclusters reagent to determine calibration factor a, wherein a=PH and PH is the peak height of the fluorescence signal; and measure a fluorescence intensity of the Au-BSA nanoclusters at an emission wavelength of 675 nm in a mixture of a water sample with a known concentration c of the GLUT composition and the Au-BSA nanoclusters reagent to determine calibration factor b, wherein

$$b = \frac{P H - a}{√ c} .$$

In another aspect, the three-way valve comprises a water sample inlet for receiving the water sample, a calibration inlet for a calibration fluid, and an outlet configured to direct the water sample or calibration fluid to the remaining components of the analyzer.

In another aspect, the analyzer further includes a reagent injector configured to inject the Au-BSA nanoclusters reagent into the 6-way valve.

In another aspect, the water sample is a calibration fluid, and wherein when the 3-way valve receives a calibration fluid, the analyzer is configured to perform a calibration cycle.

In another aspect, the analyzer has a dynamic detection range for the GLUT composition of approximately 80-2000 ppm.

In another aspect, the GLUT composition is a biocide composition and wherein the water is seawater.

In another aspect, the analyzer further comprises a heater operatively connected to the reaction coil, wherein the heater is configured to maintain the reaction coil at a temperature in the range of approximately 35-45° C. In a further aspect, the heater is configured to maintain the reaction coil at approximately 40° C.

In another aspect, the analyzer further comprises a bandpass filter configured to improve sensitivity and selectivity of the analyzer.

In another aspect, the measurement accuracy of the concentration of the GLUT composition is within ±20% of the actual GLUT concentration.

In another aspect, the analyzer is an online analyzer operatively connected to a water system and configured to perform real-time measurements of water samples from the water system. In a further aspect, the analyzer is configured to automatically switch from a slug mode to a non-slug mode if the measured concentration of the GLUT composition is below a first predetermined value and to switch from non-slug mode to slug mode if the measured concentration of the GLUT composition is above a second predetermined value.

In a further aspect, the analyzer further comprises a filter device operatively connected to the water system via tubing that receives the water sample from the water system, wherein the filter device is configured to remove debris and bubbles from the water sample; and an intermediate sample vial operatively connected to the three-way valve and configured to receive the water sample after passing through the filter device and further remove bubbles in the water sample. The filter device and the intermediate sample vial are located upstream of the three-way valve.

In a second aspect, an online analyzer operatively connected to a water system for detecting and quantifying a GLUT composition in a water sample is provided. The analyzer includes a cross-flow filter operatively connected to the water system via tubing that receives a water sample comprising a GLUT composition from the water system, wherein the filter device is configured to remove debris and bubbles from the water sample. The analyzer also includes a piston pump configured to draw in the water sample from the water system through the cross-flow filter; and a reaction vessel operatively connected to the piston pump and configured to receive the water sample via the piston pump and an Au-BSA nanoclusters reagent from a reagent supply. The water sample and the Au-BSA nanoclusters reagent are mixed and reacted in the reaction vessel and the reaction vessel is further configured to remove bubbles from the water sample and Au-BSA nanoclusters reagent mixture. The analyzer also includes a fluorescence flow cell comprising a 375 nm light-emitting diode (LED). The flow cell is configured to: receive the mixture from the reaction vessel; measure a fluorescence intensity of the Au-BSA nanoclusters in the mixture at an emission wavelength of 675 nm, wherein the Au-BSA nanoclusters comprises a maximum excitation wavelength of approximately 375 nm and a maximum emission wavelength of approximately 675 nm; and determine a presence and concentration of the GLUT composition in the water sample based on a comparison between the measured fluorescence intensity of the Au-BSA nanoclusters at the emission wavelength of 675 nm with fluorescence intensity values of calibration samples comprising Au-BSA nanoclusters and known glutaraldehyde concentrations.

In another aspect, a correlation of the fluorescence intensity of the Au-BSA nanoclusters and the concentration of the GLUT composition is based on the equation: Y=a+b\sqrt{X}, wherein Y is the peak height of the fluorescence signal, X is the concentration of GLUT composition in the water sample, and a and b are calibration factors determined using a 2-point calibration procedure. In a further aspect, to perform the 2-point calibration procedure the fluorescence flow cell is configured to: measure a fluorescence intensity of the Au-BSA nanoclusters at an emission wavelength of 675 nm in a mixture of a water sample without the GLUT composition and the Au-BSA nanoclusters reagent to determine calibration factor a, wherein a=PH and PH is the peak height of the fluorescence signal; and measure a fluorescence intensity of the Au-BSA nanoclusters at an emission wavelength of 675 nm in a mixture of a water sample with a known concentration c of the GLUT composition and the Au-BSA nanoclusters reagent to determine calibration factor b, wherein

$$b = \frac{P H - a}{√ c} .$$

In another aspect, the analyzer has a dynamic detection range for the GLUT composition of approximately 80-2000 ppm.

In another aspect, the measurement accuracy of the concentration of the GLUT composition is within ±20% of the actual GLUT concentration.

### BRIEF DESCRIPTION OF THE DRAWING FIGURES

FIGS. 1A-1C show diagrams of various aspects of an exemplary GLUT analyzer in accordance with one or more embodiments.

FIG. 1D displays the excitation and emission spectra of the Au-BSA nanoclusters in accordance with one or more embodiments.

FIG. 1E shows a chemical diagram of the fluorescence quenching of Au-BSA nanoclusters when exposed to GLUT in accordance with one or more embodiments.

FIG. 2 shows an exemplary calibration curve for GLUT measurement based on a two-point calibration method in accordance with one or more embodiments.

FIG. 3 shows an exemplary GLUT calibration curve based on seven concentrations of GLUT solutions (triplicate) in accordance with one or more embodiments.

FIGS. 4A-4C shows images of an exemplary GLUT analyzer with the cabinet doors closed (FIG. 4A); the front door open (FIG. 4B) showing the liquid flow aspects of the analyzer system; and the back door open (FIG. 4C) showing the electronic aspects of the analyzer system, in accordance with one or more embodiments.

FIG. 4D displays an image of an exemplary debubbler in accordance with one or more embodiments.

FIG. 5 displays a schematic view of Au-BSA nanocluster solution injection to the stream of the water sample or calibration solution before (left diagram) and after (right diagram) the reaction in a knitted reaction coil (center image) in accordance with one or more embodiments.

FIG. 6 displays an image of exemplary main parameter settings in a user interface of the GLUT analyzer in accordance with one or more embodiments.

FIG. 7 shows an exemplary GLUT-based biocide measurement cycle in accordance with one or more embodiments.

FIGS. 8A-8B displays graphs of GLUT-based biocide slug measurements in seawater samples with 0 ppm of biocide (FIG. 8A) and 625 ppm of the biocide BIOC31450A (FIG. 8B) as determined by the GLUT analyzer in accordance with one or more embodiments.

FIG. 9 displays a calibration curve and dynamic detection range for a GLUT-based biocide (BT1451) as determined by the GLUT analyzer in accordance with one or more embodiments.

FIG. 10 displays the results of two-point calibration and acceptance tests for a GLUT-based biocide (BT1451) solutions prepared at low, medium, and high concentrations in accordance with one or more embodiments.

FIG. 11 displays a calibration curve and dynamic detection range for a GLUT-based biocide (SM-MB-7055) as determined by the GLUT analyzer in accordance with one or more embodiments.

FIG. 12 displays the results of two-point calibration and acceptance tests for a GLUT-based biocide (SM-MB-7055) solutions prepared at low, medium, and high concentrations in accordance with one or more embodiments.

FIG. 13 displays an exemplary connection of the GLUT analyzer and a water sampling port (e.g., seawater sampling port) connected to a water system in accordance with one or more embodiments.

FIGS. 14A-14C display exemplary slug measurement curves for seawater samples with various concentrations of GLUT-based biocide (BIOC31450A), 0 ppm (FIG. 14A), 400 ppm (FIG. 14B), and 4000 ppm (FIG. 14C), in accordance with one or more embodiments.

FIG. 15 displays the results of GLUT-based biocide (BIOC31450A) slug detection measurements at a water supply plant in accordance with one or more embodiments.

FIG. 16 displays a GLUT analyzer comprising additional features for the liquid handling aspects of the analyzer in accordance with one or more embodiments.

FIG. 17 is a block diagram illustrating an exemplary configuration of an exemplary computing device operatively connected to the GLUT analyzer in accordance with one or more embodiments.

### DETAILED DESCRIPTION OF CERTAIN EMBODIMENTS

By way of overview and introduction, the present application discloses an analyzer for detecting and quantifying glutaraldehyde (GLUT)-based compounds, such as biocides, in water samples. In certain embodiments, the water samples are collected from seawater pipelines. The analyzer allows for online (e.g., operatively connected to the water system) and real-time detection and monitoring of GLUT-based biocide residuals water systems. In one or more embodiments, the analyzer has dynamic detection range of approximately 80-2000 ppm of a GLUT-based biocide formulation (or approximately 20-800 ppm of GLUT). In one or more embodiments, the measurement repeatability is less than approximately 10% variation in terms of relative standard deviation (RSD), and the measurement accuracy is within approximately ±20% of the actual GLUT-based biocide concentration. The present GLUT analyzer also provides improved ruggedness and resilience in large water treatment and injection systems, as well as improved liquid handling.

The present GLUT analyzer is effective at real-time detection of GLUT-based biocides and monitoring of the biocide residual concentration in a water system (e.g., large pipeline network), especially in remote injection wells, in order to ensure effective biocide treatment and maintain good microbial control in the water system. Specifically, in one or more embodiments, the GLUT analyzer can provide real-time monitoring of residue concentrations of GLUT biocides in downstream locations of a large pipeline network.

These and other aspects of the present analyzer and associated methods are described in further detail below with reference to the accompanied drawing figures, in which one or more illustrated embodiments and/or arrangements of the apparatus and methods are shown. The apparatus and methods of the present application are not limited in anyway to the illustrated embodiment and/or arrangement. It should be understood that the apparatus and methods as shown in the accompanying figures are merely exemplary of the apparatus and methods of the present application, which can be embodied in various forms as appreciated by one skilled in the art. Therefore, it is to be understood that any structural and functional details disclosed herein are not to be interpreted as limiting the present apparatus and methods, but rather are provided as a representative embodiment and/or arrangement for teaching one skilled in the art one or more ways to implement the present apparatus and methods. It should be understood that, as used in the present application, the term “approximately” when used in conjunction with a number refers to any number within 5% of the referenced number, including the referenced number.

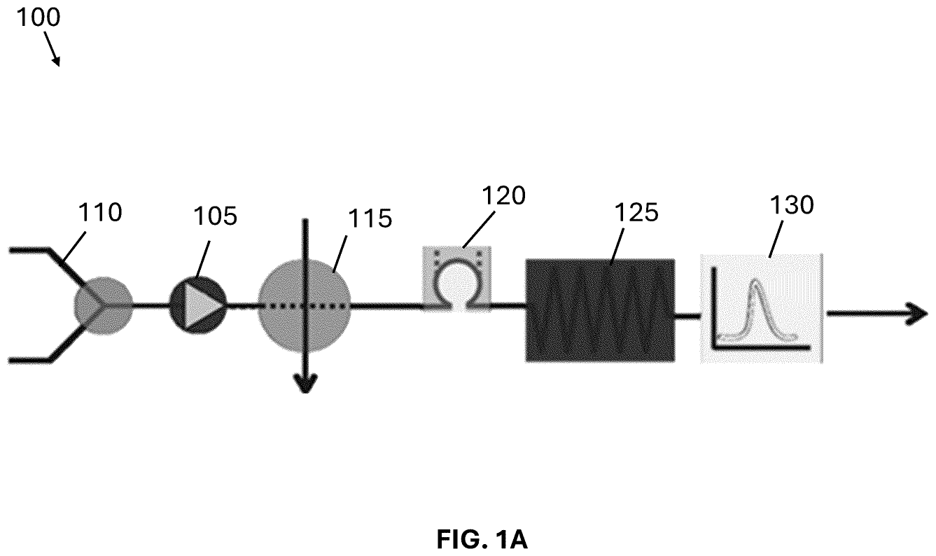

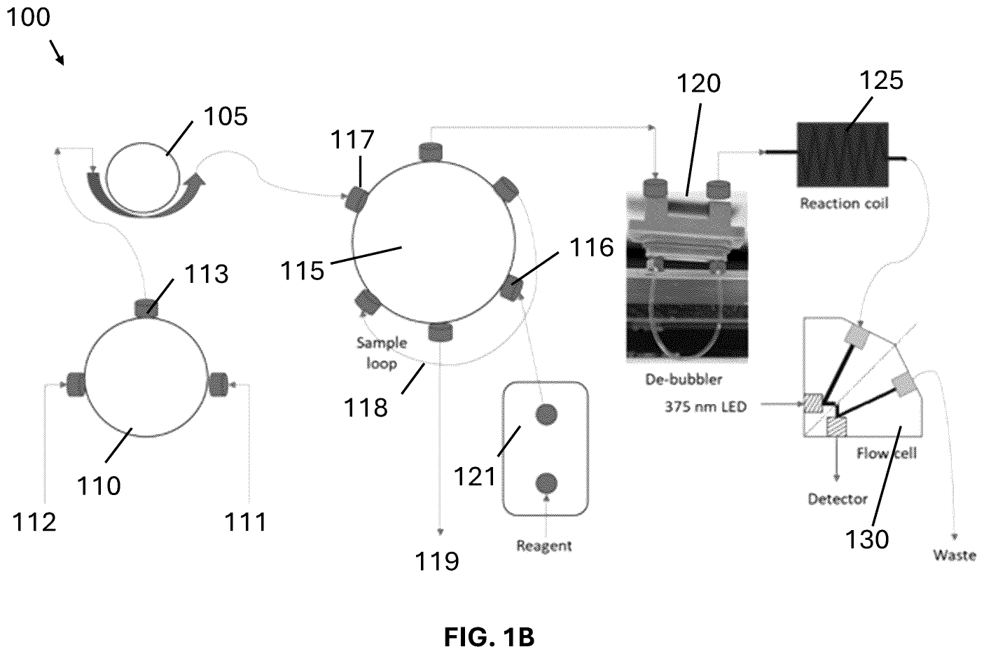

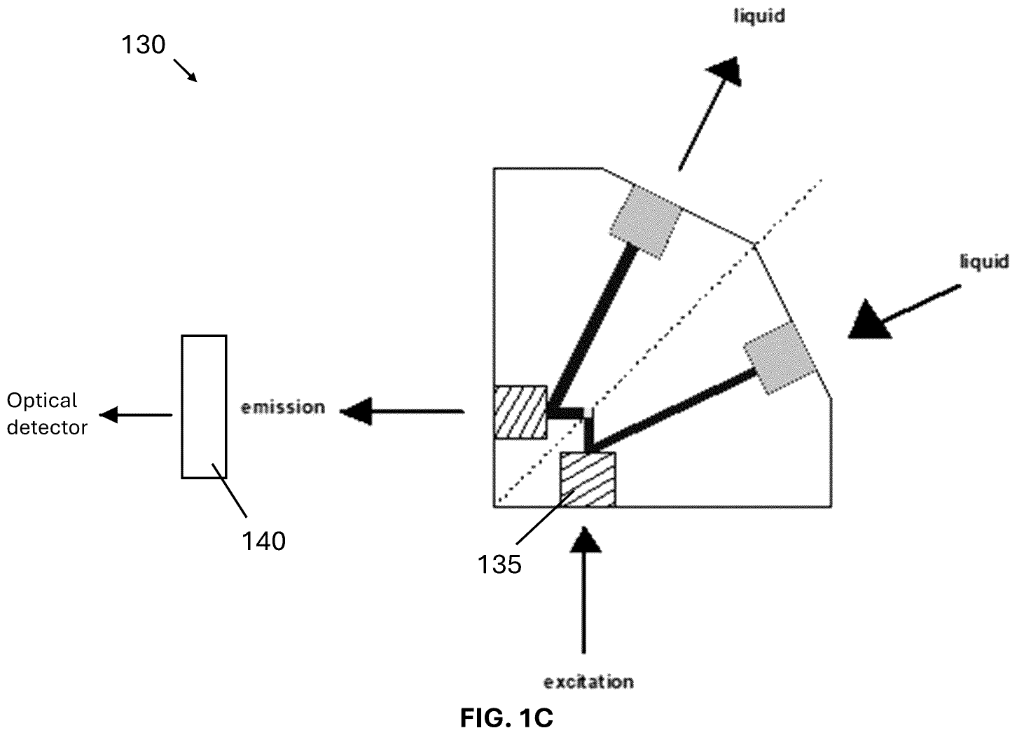

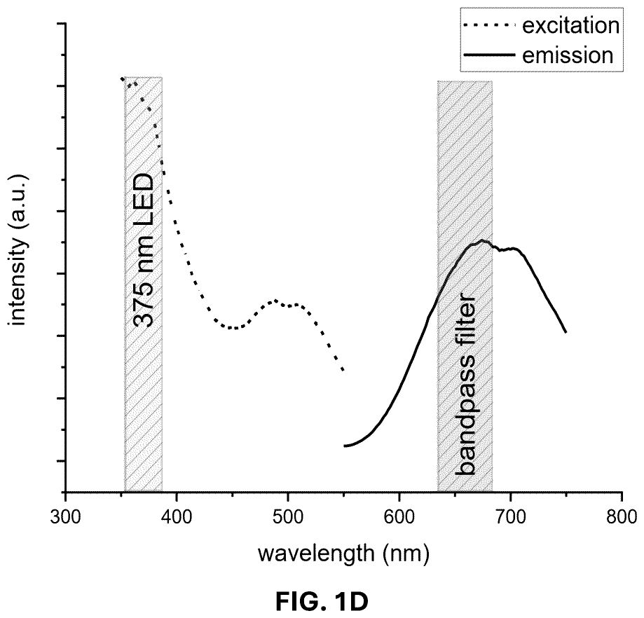

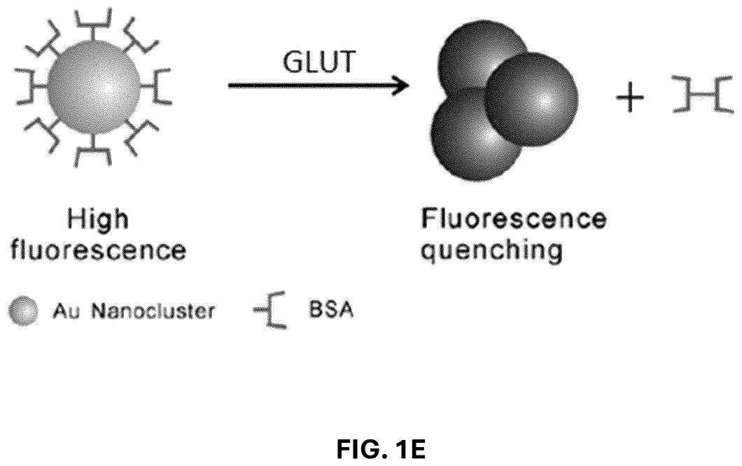

FIGS. 1A-1C show diagrams of various aspects of an exemplary GLUT analyzer in accordance with one or more embodiments. With reference now to FIGS. 1A-1B, the GLUT analyzer 100 includes a peristaltic pump 105. The pump 105 draws the water sample into the analyzer 100 via a three-way valve 110. In one or more embodiments, the pump 105 can be configured to draw in a calibration fluid into the analyzer 100 via the three-way valve 110. In certain embodiments, as exemplified in FIG. 1B, the three-way valve 110 can comprise a first inlet 111 for the water sample and a second inlet 112 for the calibration fluid, as well as an outlet 113 to direct the water sample or calibration fluid to other components of the analyzer 100. In one or more embodiments, the calibration fluid comprises of a GLUT-based composition (e.g., GLUT-based biocide formulation) with a known concentration.

The analyzer 100 also include a six-way valve or loop injector 115 that is operatively connected to the three-way valve 110 via the pump 105. During a calibration cycle or a sample measurement cycle, the peristaltic pump 105 draws a water sample or calibration fluid via the three-way valve 110 to the six-way valve or loop injector 115.

As exemplified in FIG. 1B, the loop injector 115 is also configured to receive a reagent solution comprising bovine serum albumin (BSA)-coated gold (Au) nanoparticles (Au-BSA nanoclusters) via a reagent injector 121 (or other mechanism) through an inlet 116 and the water sample or calibration fluid from the three-way valve through another inlet 117, and deliver the reagent to the water sample or calibration fluid. The loop injector 115 can also include sample loop 118 and a drain 119.

The water sample (or calibration solution) and the reagent solution flow from the six-way valve/loop injector 115 to a debubbler 120. The debubbler 120 is positioned upstream of a reaction coil 125 and is configured to remove any bubbles from the de-pressurized water sample.

With continued reference to FIGS. 1A-1B, the water sample (or calibration solution) and the reagent solution flow from the debubbler 120 to the mixing coil or reaction coil 125 for mixing the reagent solution with the water sample or calibration solution. The reagent and the water sample (or calibration solution) are thoroughly mixed in the reaction coil 125 to cause a chemical reaction. Specifically, the GLUT (if present) in the water sample or calibration solution, will react with the Au-BSA nanoclusters in the reagent solution, as explained in further detail below.

The analyzer 100 also includes an optical detector 130 operatively connected to the reaction coil 125 and configured to receive the reaction mixture from the reaction coil 125. In one or more embodiments, the optical detector 130 comprises a fluorescence flow cell, as shown in FIG. 1C in accordance with one or more embodiments. As exemplified in FIG. 1C, in one or more embodiments in the fluorescence flow cell 230, the mixture comprising the reagent solution (Au-BSA nanoclusters) and the water sample is exposed to the light emitted by a 375 nm light-emitting diode (LED) 135 to produce fluorescence. The 375 nm LED 135 is used for excitation, and the fluorescence emission at 675 nm is measured. In one or more embodiments, a bandpass filter 140 (e.g., 658±40 nm bandpass filter) is used to improve sensitivity and selectivity (e.g., measuring fluorescence signals in the range of approximately 630-680 nm). FIG. 1D displays the excitation and emission spectra of the Au-BSA nanoclusters in accordance with one or more embodiments. The net fluorescence signal (peak height, PH) is calculated by subtracting the baseline (BL) signal from the fluorescence maximum.

In one or more embodiments, when the fluorescence signals of the Au-BSA nanoclusters is collected by the fluorescence flow cell 130, it is transported via an optical cable to a data processor (e.g., microcontroller) to process the signals and measure the fluorescence signal intensity of the Au-BSA nanoclusters. After the fluorescence of the Au-BSA nanoclusters has been measured and the concentration of GLUT in the sample has been determined, the mixture of the water sample and the Au-BSA nanoclusters is passed out of the flow cell 130 and can be disposed of as waste.

The determination of the presence and concentration of GLUT in the sample is based on the correlation of the fluorescence signal of Au-BSA nanoclusters and GLUT concentrations. Specifically, Au-BSA nanoclusters show strong fluorescent properties at maximum excitation (EX) wavelength of approximately 375 nm and a maximum emission (EM) wavelength of approximately 675 nm. The GLUT detection by the present analyzer is thus based on fluorescence quenching of Au-BSA nanoclusters when exposed to GLUT (see FIG. 1E). More specifically, the fluorescence flow cell 130 is configured to measure a fluorescence intensity of the Au-BSA nanoclusters in the mixture at an emission wavelength of 675 nm, where the Au-BSA nanoclusters comprises a maximum excitation wavelength of approximately 375 nm and a maximum emission wavelength of approximately 675 nm. The flow cell 130 is then configured to determine a presence and concentration of the GLUT in the water sample based on a comparison between the measured fluorescence intensity of the Au-BSA nanoclusters at the emission wavelength of 675 nm with fluorescence intensity values of calibration samples comprising Au-BSA nanoclusters and known glutaraldehyde concentrations.

The correlation of fluorescence signal of Au-BSA nanoclusters and GLUT concentrations fits into the following equation: Y=a+b\sqrt{X}, where Y is the peak height of fluorescence signal; X is the concentration of GLUT biocide (ppm); and the calibration factors a and b are determined using a two-point calibration procedure.

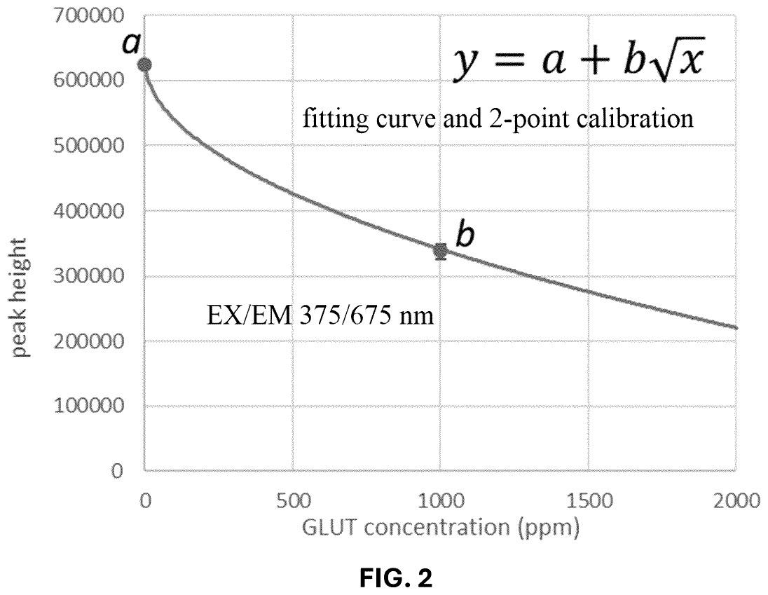

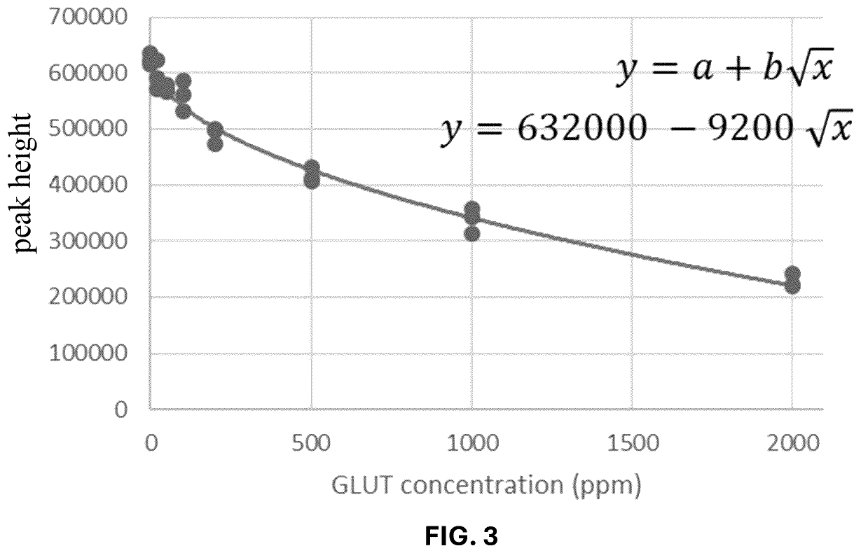

An exemplary calibration curve for GLUT measurement based on two-point calibration is shown in FIG. 2. While flowing through the fluorescence flow cell, the fluorescence maximum of the Au-BSA reagent solution is measured at 675 nm. The net fluorescence peak signals at 0 ppm (1st point) and another known concentration (2nd point) of the GLUT-based composition (e.g., biocide) are used to calculate the calibration factors a and b, and the calibration equation (FIG. 2). For unknown samples, the net fluorescence peak signals are used to determine the concentration of GLUT-based composition in the water sample using the calibration equation. An exemplary GLUT calibration curve and fitting equation based on seven GLUT concentrations (and a blank) is shown in FIG. 3.

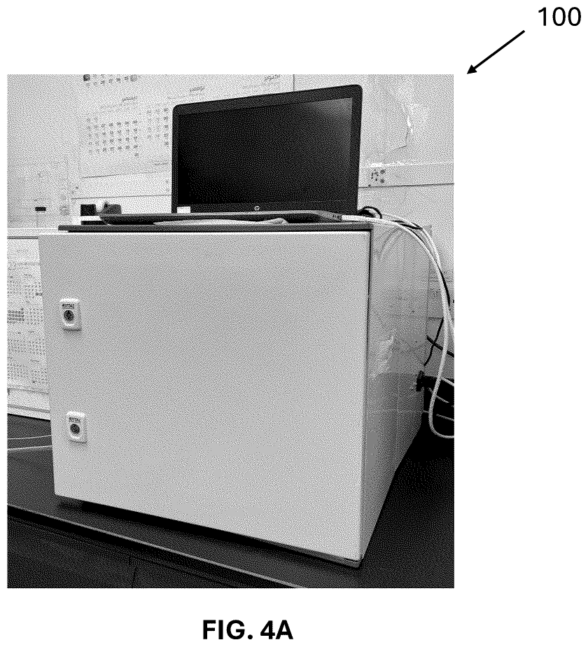

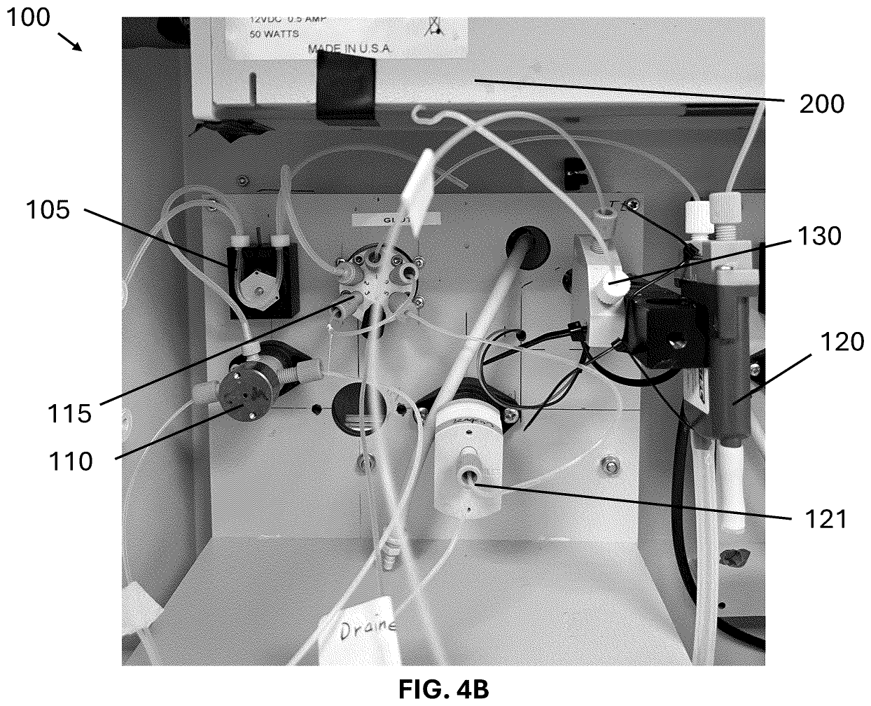

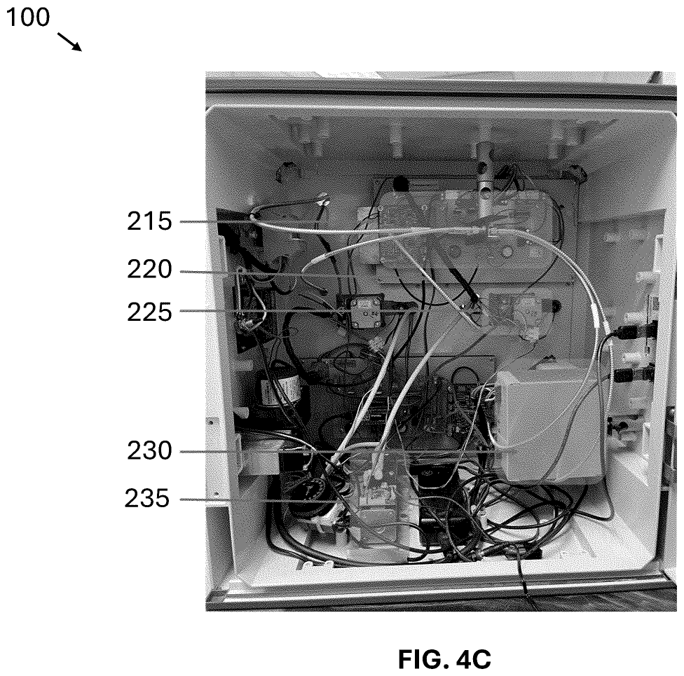

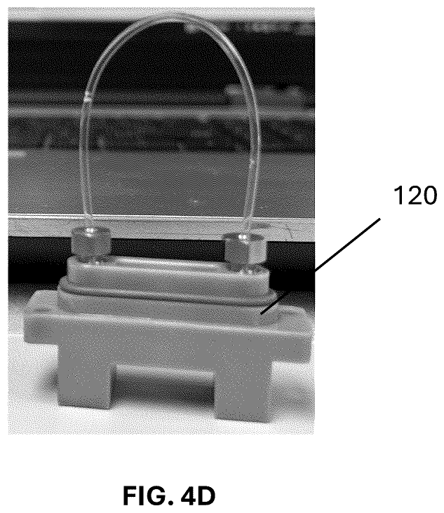

In one or more embodiments, the GLUT analyzer of the present application can be constructed using an enclosure or cabinet for protection and temperature control. For example, in one or more embodiments the GLUT analyzer can be constructed using mounted cabinets that, in certain instances, can be arranged back-to-back. FIGS. 4A-4C show images of an exemplary GLUT analyzer with the cabinet doors closed (FIG. 4A), the front door open (FIG. 4B) showing the liquid flow aspects of the analyzer system, and the back door open (FIG. 4C) showing the electronic aspects of the analyzer system.

As exemplified in FIG. 4B, the front cabinet houses the liquid flow aspects of the system, including the processing fluids (e.g., water sample or calibration fluid, and reagent solution), the detector 130 (including the flow cell), debubbler 120, reaction coil 125, 3-way and 6-way valves (110 and 115, respectively), reagent injector 121, and peristaltic pump 105. In one or more embodiments, the liquid flow aspects of the analyzer can further include a heater 200 that encompasses the reaction coil 120. In one or more embodiments, the heater 200 can be configured to be maintained at a temperature in the range of approximately 35-45° C. In at least one embodiment, the heater 200 can be configured to be maintained at a temperature of approximately 40° C.

As exemplified in FIG. 4C, the back cabinet can include the electronic aspects of the analyzer, including the control printed circuit boards (PCBs) 215, the optical fiber 220, the pump engine 225 for the peristaltic pump, a power supple 230, and a pump 235 for the debubbler. An image of an exemplary debubbler 120 is shown in FIG. 4D in accordance with one or more embodiments.

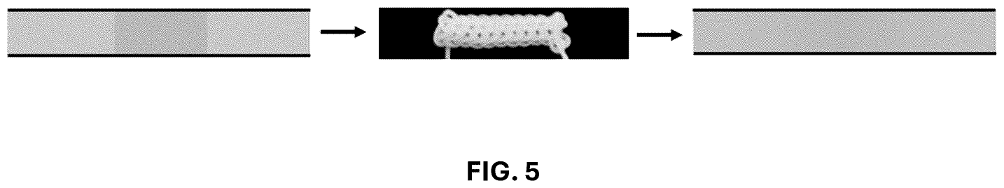

As previously mentioned, during a calibration cycle or a sample measurement cycle, the peristaltic pump draws in the calibration solution (e.g., solution having known concentration of GLUT) or the water sample via a 3-way valve to the 6-way valve. The reagent solution (AU-BSA nanocluster solution) can then be injected into the calibration solution or water sample in the 6-way valve via an injector (e.g., micropump). For example, in at least one embodiment, a micropump can injects 50 μl of AU-BSA nanocluster reagent solution into the GLUT-containing water sample or calibration solution. FIG. 5 displays a schematic view of the Au-BSA nanocluster reagent solution injected into the stream of the calibration solution or water sample, before (left diagram) and after (right diagram) the mixture of the Au-BSA nanocluster solution and the water sample (or calibration solution) enter a knitted reaction coil (middle image). The AU-BSA nanocluster reagent solution and the GLUT-containing water sample first pass through the debubbler (e.g., 480 μl capacity, with −0.99 barg vacuum pump) for removing bubbles in the water sample. The de-gassed calibration solution or GLUT-containing water sample and the reagent solution are then mixed thoroughly and react at 40° C. when they move through the reaction coil, which in certain embodiments can be approximately 1 meter in length. The de-gassed mixture then flows through the fluorescence flow cell of the detector to a waste container.

In one or more embodiments, the GLUT analyzer of the present application is configured to operate on freshwater samples or salt water (seawater) samples. In one or more embodiments, the GLUT analyzer is configured to operate on seawater samples having up to approximately 5.5% salinity. In at least one embodiment, the GLUT analyzer is configured to operate on seawater samples having approximately 7-15% salinity.

In certain embodiments, the GLUT analyzer is designed to operate under lab conditions for temperature control. For example, the GLUT analyzer can be installed in the other locations (e.g., a remote area) with an enclosure and air conditioning.

In one or more embodiments, the water system that the GLUT analyzer receives the water sample from is a pressurized water system, and thus the water pressure of the water sample needs to be regulated down (de-pressurized) to be at or near approximately 1 atm before entering the GLUT analyzer.

Exemplary parameters for the GLUT analyzer of the present application are shown in Table 1 below.

TABLE 1
Specification of prototype GLUT analyzer.
Specifications for GLUT analyzer
Reagent

| Concentration | See attached “Protocol for the production of gold-BSA | |
|---|---|---|
| | nanoparticles reagent” | |
| Reagent injection volume | 50 | μl |

| | |
|---|---|
| Shelf life | At least one month at 4° C. |

Sampling

| Sample flow rate | 150 | μl/min |
|---|---|---|
| Sample temperature | <40° | C. |
| Sample pressure | normal pressure (1 atm) | |
| Max particles | 0.2 | μm |

Measurement

| Detection method | Fluorescence | |
|---|---|---|
| Wavelength | EX 375 nm/EM 675 nm | |
| Maximum measurement | 4 times per hour | |
| frequency | | |
| Calibrator* | GLUT-based formulation | |
| Detection range | 20-800 mg/L GLUT | |
| Repeatability (RSD) | <10% | |
| Accuracy | ±20% @ 20 ppm | |
| | ±20% @ 400 ppm | |
| | ±20% @ 800 ppm | |
| Measurement time | 9 | minutes |

Connectivity

| | |
|---|---|
| Communication | Serial over USB |
| Hardware Accessibility | Cabinet lockable |
| Software Accessibility | Optional password |

General

| | | |
|---|---|---|
| Reagent + calibrator | 4° | C. |

| | |
|---|---|
| temperature | |
| Debubbler/degasser | Yes |
| Power supply | 110-120 V/200-240 V/300 VA/50/60 Hz |
| Housing Material | ABS steel |
| Dimensions H × W × D | 50 × 30 × 60 cm |
| Weight | Typical 15 kg |

*Concentration of analyte (GLUT) varies in the formulations.

### 2-Point Calibration Procedure

As mentioned above, the correlation of fluorescence signal of Au-BSA nanoclusters and GLUT concentrations fits into the following equation: Y=a+b\sqrt{X}, where Y is the peak height of fluorescence signal; X is the concentration of GLUT biocide (ppm); and the calibration factors a and b are determined using a two-point calibration procedure.

In one or more embodiments, the GLUT analyzer of the present application is connected to the source of the water sample or a line connected the water sample source, and water sample stream flows through the analyzer continuously. During a calibration cycle, the reagent (Au-BSA nanocluster solution) is injected into a water sample containing no GLUT to form a mixture, and the mixture passes through the debubbler to remove bubbles and then into the reaction coil for thorough mixing. The mixed solution passes to the fluorescence flow cell, where the fluorescence of the Au-BSA nanoclusters is measured at 675 nm. The measurement signals (peak height, PH) are calculated by subtracting the average baseline (BL) signals. The calibration factor a is then determined. This process to determine factor a is called CAL0: a=PH.

Next, a water sample containing a known concentration of a GLUT-based formulation (i.e., the calibration fluid) is prepared, e.g., a 625 ppm sample. The analyzer carries out the same measurement process, and obtains a fluorescence PH signal of Au-BSA nanoclusters. The calibration factor b is now determined. This process to determine factor b is called CAL1:

$$b = \frac{P H - a}{√ 625} .$$

It should be understood that 625 ppm is an exemplary known concentration for the calibration fluid, and in other embodiments, the calibration fluid can have a different known concentration “c” of GLUT, such that the generic equation for determining factor b is:

$$b = \frac{P H - a}{√ c} .$$

The calibration factors a and b can then be automatically updated via the data processor into software of the analyzer, and used in the future measurements of unknown water samples to determine the concentration of a GLUT-based formulations in the samples.

### Measurement Procedure for Unknown Samples

After the analyzer has been calibrated and the factors a and b are determined, and the correlation of fluorescence PH and biocide concentration is established. When the sampling tube of the GLUT analyzer is connected to an unknown sample (such as a sea water sample), it carries out the same measurement process as described above, and obtains a fluorescence PH. The analyzer, via the data processor can then determine the concentration of the GLUT-based formulation (e.g., GLUT-based biocide) in the unknown sample using PH and the correlation equation:

$$Conc . ( ppm ) = {( \frac{P H - a}{b} )}^{2}$$

Exemplary measurement conditions of the GLUT analyzer are summarized below in Table 2. With the peristaltic pump, the sample flow rate is maintained at approximately 140-150 μl/min in accordance with one or more embodiments. In one or more embodiments, the Au-BSA reagent solution is prepared, and approximately 50 μl of the reagent solution is injected into the sample stream by a micropump. To counter the potential variation of the injection volume due to various reasons, the micropump can be set to perform more than 1 stroke of injection to fill the 50 μl sample loop, before the reagent solution is delivered to the sample stream. Mixing and reaction of GLUT and the reagent solution takes place in the reaction coil at approximately 40° C. In one or more embodiments, the reaction coil can be approximately 1-2 meters in length. In at least one embodiment, the reaction coil can be approximately 1 meter in length. In one or more embodiments, the fluorescence of the Au-BSA nanoclusters solution is measured for 9 minutes.

TABLE 2
Measurement conditions for prototype GLUT analyzer.

| Parameter | value | |
|---|---|---|
| Reaction temperature | 40° | C. |
| Sample flow rate | 150 | μl/min |
| Injection volume (reagent) | 50 | μl |
| Reagent | Au-BSA nanoparticles | |
| Length of reaction coil | 1 | m |
| Measurement time | 9 | min* |

| | |
|---|---|
| Fluorescence measurement | EX/EM 375/675 nm |

*9 min measurement time can be reduced with more appropriate size of the de-bubbler. For instance, a 100-μl size de-bubbler only require 6 min measurement.

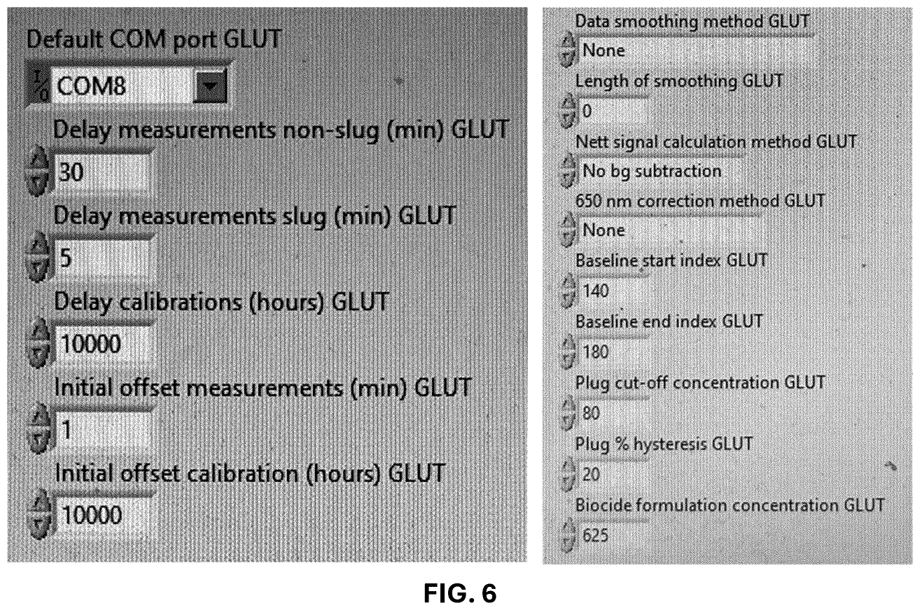

An example of the main parameter settings in the user interface of the present GLUT analyzer in is shown in FIG. 6. For example, “delay measurements non-slug (min)” refers to the time between a measurement and the next measurement, when the system is not in slug mode. The term “delay measurements slug (min)” refers to the time between a measurement and the next measurement, when the system is in slug mode. The term “delay calibrations (hours)” refers to the time between a calibration and the next calibration. The term “initial offset measurements (min)” refers to the time between starting the program and the first measurement. The term “Initial offset calibrations (hours)” refers to the time between starting the program and the first calibration, and the term “baseline start & end index” refers to the indices over which to calculate the baseline. The term “plug cut-off concentration and hysteresis,” for example at 80 ppm and 20%, refers to the system being able to go from non-slug to slug mode when the concentration is above x ppm+y % (e.g., 96 ppm) or go from slug to non-slug mode when the concentration is below x ppm−y % (e.g., 64 ppm). Finally, the term “Biocideformulation concentration” refers to concentration of the GLUT-based formulation (e.g., biocide) in the calibration solution.

“Biocide batch treatment” is common in oil and gas industry and refers to biocide treatments in which the biocide is only injected into the water system at a pre-determined frequency (e.g., weekly) and for a pre-determined period of time (e.g., 2 hours). To prevent waste or depletion of the reagent, in certain embodiments the GLUT analyzer of the present application can be configured automatically switch between “slug” and “non-slug” mode depending on the concentration of GLUT biocide detected in the water system, where “slug” refers to the biocide batch treatment. For example, if the concentration of the GLUT biocide is below a cut-off amount (e.g., 64 ppm), the GLUT analyzer can be switch to non-slug mode (i.e. no biocide in the water system). Therefore, the analyzer will operate in non-slug mode with longer measurement intervals. On the other hand, if the GLUT analyzer detects the GLUT biocide above a cut-off amount (e.g., 96 ppm), the analyzer will operate in slug mode (i.e., biocide present in the water system). The analyzer will then measure the sample at shorter intervals in order to catch more data points before the slug passes the analyzer location.

In short, “slug” mode refers to when there is a biocide slug in the water system, and the GLUT analyzer will measure more frequently to catch more data points before the slug passes. “Non-slug” mode means there is no biocide in the water system, and the GLUT analyzer will measure less frequently to prevent unnecessary waste of the reagent. The GLUT analyzer can switch the measurement mode automatically depending on the measured biocide concentration and the cut-off setting.

### Biocide Slug Measurement Cycle

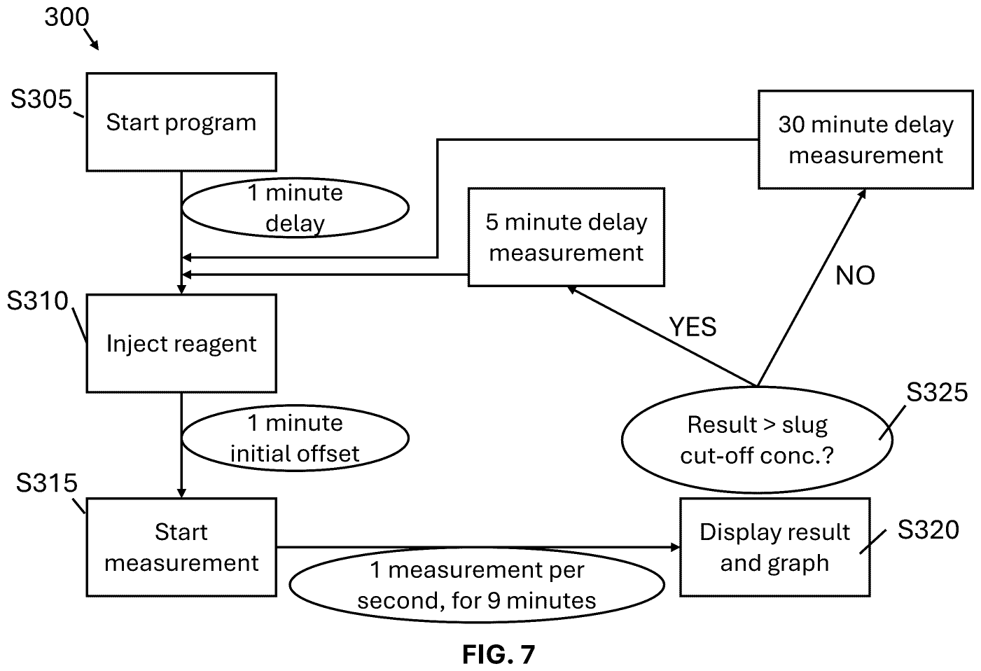

In accordance with one or more embodiments, an exemplary biocide slug measurement cycle is depicted in FIG. 7. Specifically, the flow diagram of FIG. 7 depicts a typical measurement cycle 300 for biocide slug in the source of the water sample. With reference to FIG. 7, at step S305 the cycle begins. At step S310, one minute after starting the program, 50 μl reagent solution is injected into the sample stream. Then the analyzer waits for 1 min for the LED to stabilize and at step S315 starts the fluorescence measurement, one measurement per second for a total of nine minutes. Then, at step S320 the biocide concentration is calculated using the correlation equation, and compares to the biocide plug cut-off concentration (e.g., 80 ppm±20%), and the result can be displayed.

At step S325, the analyzer is configured to make a decision—specifically, to determine the analyzer's measurement mode (slug vs. non-slug mode)—for the next measurement. In one or more embodiments, the difference between these two modes is the delay time for the next measurement. Non-slug mode has a longer delay time (e.g., 30 min before next measurement), while the slug mode has a shorter delay time (e.g., 5 min) (see e.g., parameters FIG. 6). The operator can set a longer delay time to reduce the consumption of the reagent when the GLUT-based biocide slug has not arrived at the detection site, for example. On the other hand, the operator can set a shorter delay time for the next measurement to allow the analyzer to take more measurements before the GLUT-based biocide slug passes the detection site. A typical biocide slug measurement cycle takes 15 minutes (e.g., 1 minute initial offset, 9 minute measurement, and 5 minute delay for next measurement). The five minute delay allows the analyzer to clear the liquids in the flow system from the previous measurement, and therefore, minimize the carry-over signals, improving the quality of baseline signals—in other words, the signals will be more stable with less variation.

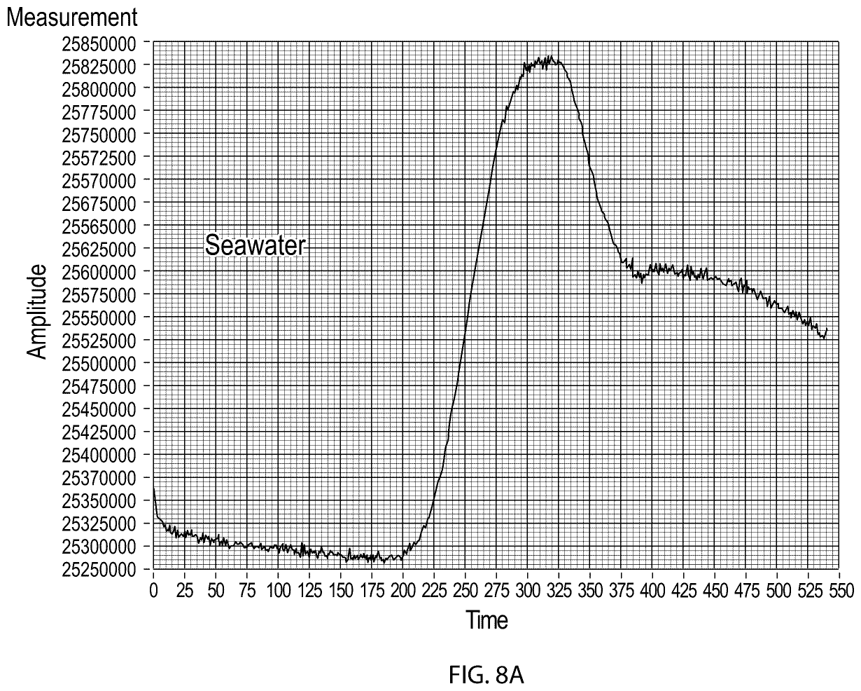

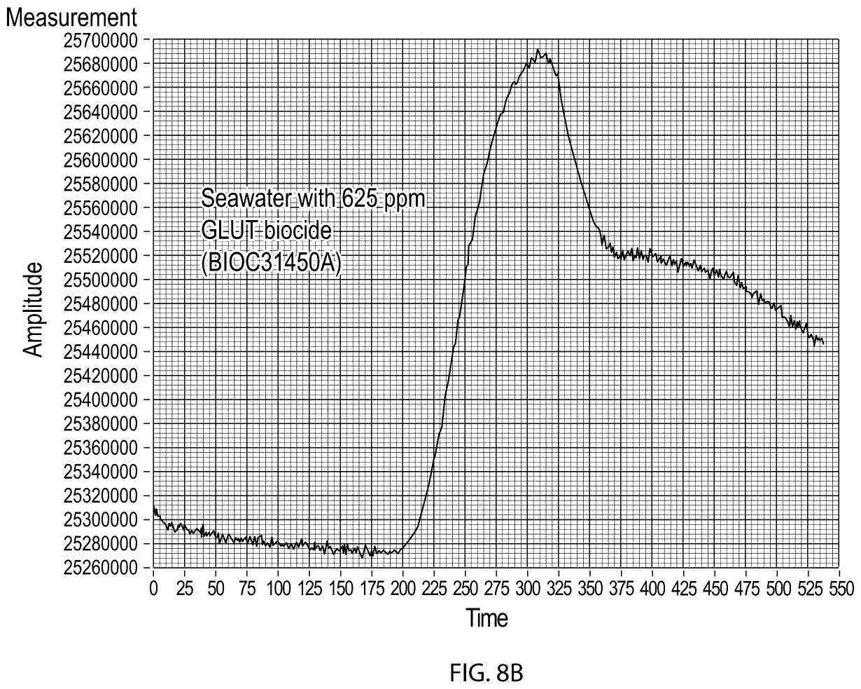

Example graphs for GLUT measurement on sea water samples containing 0 ppm and 625 ppm biocide (BIOC31450A), respectively, are shown in FIGS. 8A and 8B. Using the exemplary parameter settings in FIG. 6 as an example, the baseline signal is calculated by taking the average of measurement points from 140 to 180 seconds. The peak height (PH) signal is calculated by subtracting the baseline signal from the fluorescence maximum at 675 nm. It is noted that while these time values are optimized values for the GLUT analyzer in certain embodiments, it should be understood that these values can be modified based on sample flow rate, the distance between sampling point and the flow cell, the size of de-bubbler, and delay time for next measurement, for example.

### Acceptance Tests of the GLUT Analyzer

An acceptance test of the analyzer in accordance with one or more embodiments was performed to confirm hardware and software functionalities, and determine the dynamic measurement range, measurement repeatability (Relative Standard Deviation, RSD), and accuracy using commercial biocide products. The predefined criteria for the acceptance tests are <10% variation (RSD) for measurement repeatability and within ±20% for measurement accuracy when compared to the calculated value (see Table 3, below). Two GLUT-based biocide products were selected for the acceptance tests (see Table 4, below). It is noted that the biocide SM-MB-7055 also contains 5-10% quaternary ammonium compounds, benzyl-C12-16-alkyldimethyl, chlorides (Quats).

TABLE 3
Predefined criteria for the acceptance
tests of prototype GLUT analyzer.

| Criteria | | Prototype GLUT analyzer |
|---|---|---|
| Detection range | as active | 20-800 ppm GLUT |
| | as product | 80-2000 ppm product |
| Repeatability | peak height | <10% |
| (RSD) | | |
| Accuracy (%) | as active | ±20% @ 20 ppm |
| | | ±20% @ 400 ppm |
| | | ±20% @ 800 ppm |
| | as product | ±20% @ 100 ppm |
| | | ±20% @ 1000 ppm |
| | | ±20% @ 2000 ppm |

TABLE 4
Biocide products used for acceptance tests.

| | | Quaternary ammonium compounds, benzyl- |
|---|---|---|
| Biocide name | GLUT | C12-16-alkyldimethyl, chlorides (Quats) |
| BT1451 | 20-30% | |
| SM-MB-7055 | 10-30% | 5-10% |

The neat biocide products are considered as 100% or 1,000,000 ppm, and a series of dilutions was prepared in Qurayyah seawater (QSW) for the calibration curve, dynamic detection range, 2-point calibrations, and acceptance tests at low, medium and high concentrations (see Table 5, below). The concentrations of the active ingredient (GLUT) in Table 5 were estimated based on the average of the active ingredient's concentration range (Table 4). Biocide BT1451 and SM-MB-7055 was diluted to 80-4000 ppm of biocide product (20-1000 ppm GLUT active), diluted to 80-4000 ppm of biocide product (20-1000 ppm GLUT active in BT1451, and 16-800 ppm GLUT active in SM-MB-7055). The concentration of the prepared biocide samples based on the product dilution is designated as the calculated concentration. The low-medium-high concentrations for the acceptance tests are in the range of 100 to 2000 ppm of biocide products (20-500 ppm of GLUT active), which cover the concentration range in the biocide injection practices. The measurement conditions in the acceptance tests were shown in Table 2, above.

TABLE 5
Preparation of biocide products for various tests.

| | | product | GLUT | SM-MB- | | product | GLUT |
|---|---|---|---|---|---|---|---|
| BT1451 | DF* | ppm | ppm | 7055 | DF | ppm | ppm |
| Calibration | 12,500 | 80 | 20 | Calibration | 12,500 | 80 | 16 |
| curve with | 5,000 | 200 | 50 | curve with | 5,000 | 200 | 40 |
| detection | 1,250 | 800 | 200 | detection | 1,250 | 800 | 160 |
| range | 500 | 2000 | 500 | range | 500 | 2,000 | 400 |
| | 250 | 4000 | 1000 | | 250 | 4,000 | 800 |
| 2-point Cal. | 1,600 | 625 | 156 | 2-point Cal. | 1,600 | 625 | 125 |
| Low | 10,000 | 100 | 25 | Low | 10,000 | 100 | 20 |
| Medium | 1,000 | 1,000 | 250 | Medium | 1,000 | 1,000 | 200 |
| High | 500 | 2,000 | 500 | High | 500 | 2,000 | 400 |

*DF: dilution factor

Results of Acceptance Tests with Biocide Product BT1451

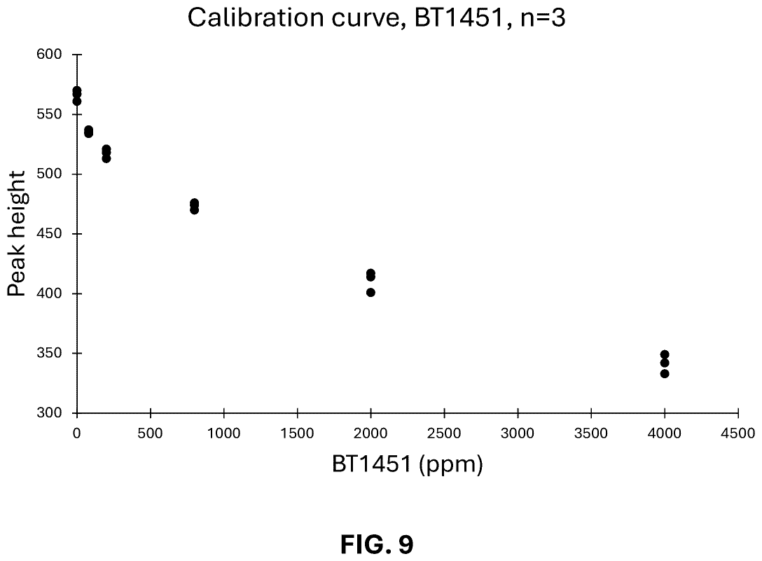

The dynamic detection range results for biocide BT1451 are shown in calibration curve of FIG. 9. The upper detection limit for BT1451 biocide is at least 4000 ppm, which is approximately 1000 ppm of GLUT active (assuming the average GLUT concentration in the formulation is 25%). The PH measurement repeatability (RSD) is between 0.3%-2.3% among the triplicate measurements at the concentration up to 4000 ppm of BT1451 biocide (see Table 6, below), meeting the smaller than 10% criterion (see Table 3, above).

TABLE 6
Peak Height (PH) measurement repeatability
(RSD) of BT1451 biocide.

| | |
|---|---|
| Calculated ppm, | Peak height (PH) |

| BT1451 | mean, n = 3 | Stdev | RSD (%) |
|---|---|---|---|
| 0 | 566.0 | 4.6 | 0.8% |
| 80 | 535.3 | 1.5 | 0.3% |
| 200 | 517.3 | 4.0 | 0.8% |
| 800 | 473.3 | 3.1 | 0.6% |
| 2000 | 410.7 | 8.5 | 2.1% |
| 4000 | 341.3 | 8.0 | 2.3% |

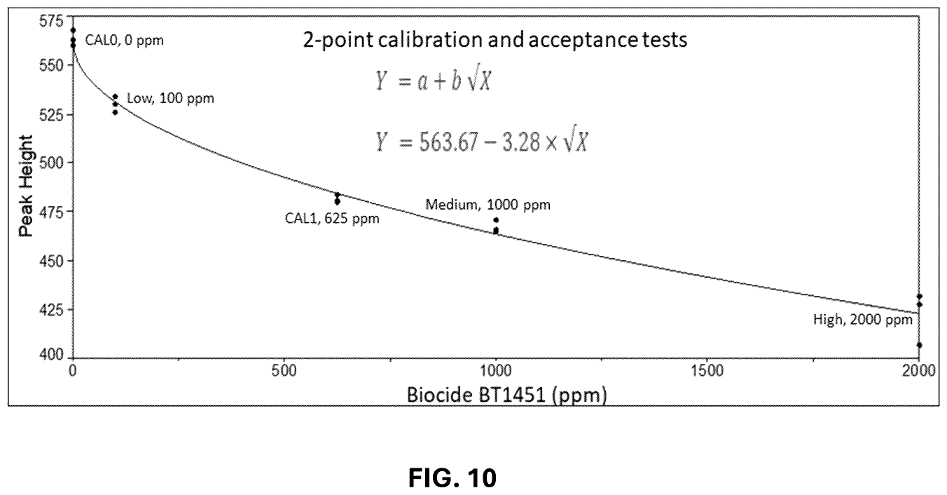

A 2-point (0 ppm and 625 ppm BT1451) calibration procedure was used to determine the factor a and b, and the calibration equation (FIG. 10). Three freshly prepared BT1451 solutions at low, medium, and high concentrations (100, 1000, and 2000 ppm) were measured in triplicate using prototype GLUT analyzer to determine the measurement repeatability (PH) and accuracy (ppm) (see FIG. 10; Table 7, below). The results in Table 7 showed that the measurement repeatability (RSD) of PH is between 0.7% and 3.2% among the triplicate measurements at low-medium-high concentration range, which meets the <10% criterion (see Table 3). The relative errors of the measured vs. calculated values (ppm) at low-medium-high concentration of BT1451 biocide were between 5.4% and

−13.7%. This also meets the smaller than ±20% criterion (see, Table 3). In conclusion, the measurement of BT1451 biocide passed the predefined criteria for the acceptance tests.

TABLE 7
Measurement repeatability (PH) and accuracy of BT1451 biocide.

| Accep- | Calcu- | Peak height (PH) | | | | Accuracy (%) |
|---|---|---|---|---|---|---|
| tance | lated | mean, | | RSD | Measured | Measured |
| tests | ppm | n = 3 | stdev | (%) | ppm | vs calc. |
| 2-point | 0 | 563.7 | 4.0 | 0.7% | | |
| cal. | 625 | 481.7 | 2.1 | 0.4% | | |
| Low | 100 | 530.0 | 4.0 | 0.8% | 105.4 | 5.4% |
| Medium | 1000 | 467.3 | 3.2 | 0.7% | 862.6 | −13.7% |
| High | 2000 | 422.3 | 13.4 | 3.2% | 1856.7 | −7.2% |

Results of Acceptance Tests with Biocide Product SM-MB-7055

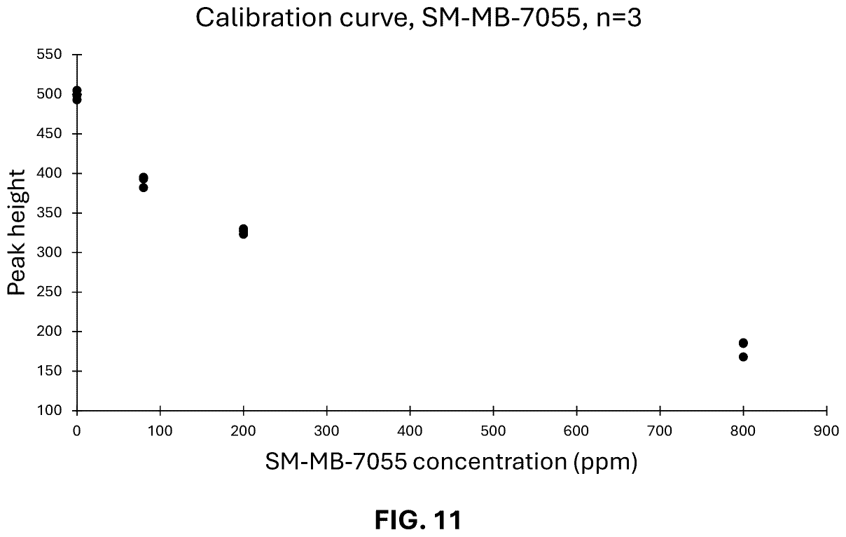

The dynamic detection range for biocide SM-MB-7055 is shown in the calibration curve of FIG. 11. The upper detection limit for SM-MB-7055 biocide is at least 800 ppm, which is approximately 160 ppm of GLUT active (assuming the average GLUT concentration in the formulation is 20%). The PH measurement repeatability (RSD) is between 1.2%-5.6% among the triplicate measurements at the concentration range of up to 800 ppm SM-MB-7055 biocide (Table 8, below), meeting the smaller than 10% criterion (see Table 3, above). Table 8. PH measurement repeatability (RSD) of SM-MB-7055 biocide.

TABLE 8
PH measurement repeatability (RSD) of SM-MB-7055 biocide.

| | |
|---|---|
| Calculated ppm, | Peak height (PH) |

| SM-MB-7055 | mean, n = 3 | Stdev | RSD (%) |
|---|---|---|---|
| 0 | 499.0 | 6.0 | 1.2% |
| 80 | 390.0 | 7.0 | 1.8% |
| 200 | 326.7 | 3.5 | 1.1% |
| 800 | 179.7 | 10.1 | 5.6% |

However, fluorescence measurement at the concentration of 2000 and 4000 ppm of SM-MB-7055 (or 400 and 800 ppm of GLUT) was not successful as the signals cannot be distinguished from the background signal. In addition to the active ingredient GLUT, this biocide also contains 5-10% Quats in the formulation (Table 4). The results of narrower dynamic range (up to 800 ppm SM-MB-7055 or 160 ppm GLUT) indicated that the presence of a significant amount of Quats in the GLUT-based biocide formulation affects the measurement at higher biocide concentration. To mitigate the impact of Quats on GLUT measurement, in at least one embodiment, a 2-fold dilution of the sample is sufficient to measure up to 2000 ppm SM-MB-7055 (or 400 ppm GLUT), which covers the concentration range in the biocide injection practices.

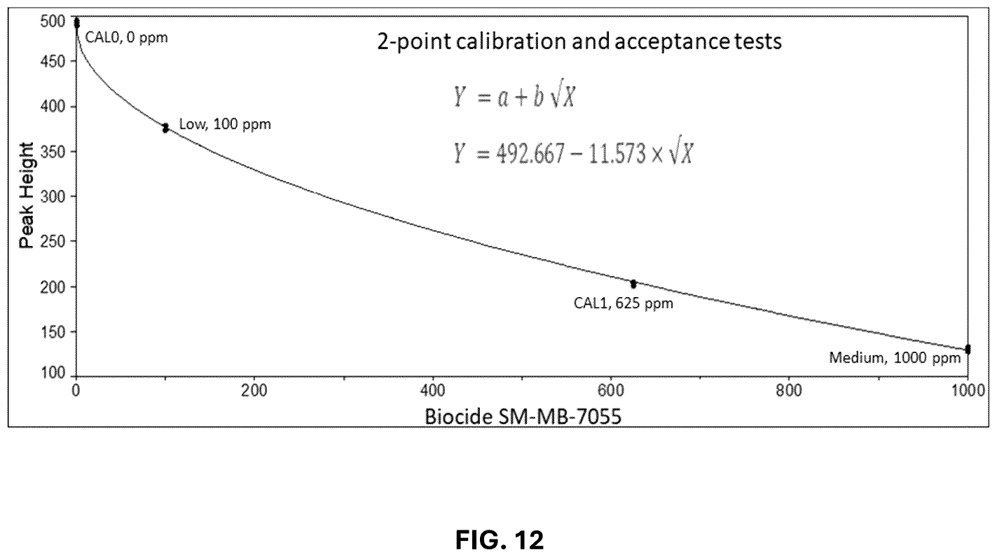

A 2-point calibration procedure (0 ppm and 625 ppm SM-MB-7055) was used to determine the factor a and b, and the calibration equation (see FIG. 12). Three freshly prepared SM-MB-7055 solutions at low, medium, and high concentrations (100, 1000, and 2000 ppm) were measured in triplicate using the GLUT analyzer in accordance with one or more embodiments to determine the measurement repeatability (PH) and accuracy (see FIG. 12; Table 9, below).

The results in Table 9 showed that the measurement repeatability (RSD) of PH is between 0.8% and 2.0% among the triplicate measurements at low and medium concentration range, which meets the <10% criterion (see Table 3, above). The relative errors of the measured vs. calculated values (ppm) at low and medium concentration of SM-MB-7055 are within ±3.0%. This also meets the smaller than ±20% criterion (see Table 3, above). However, the measurement at high concentration (2000 ppm) of SM-MB-7055 was not successful due to the presence of 5-10% of Quats in the formulation (Table 4). Therefore, the dynamic measurement range for this particular biocide is 0-1000 ppm neat product.

In conclusion, the measurement of biocide SM-MB-7055 passed the predefined criteria for the acceptance tests at up to 1000 ppm of neat product. Above this concentration, the resulting peak in fluorescence was too low to obtain a reliable measurement. It is estimated from the data of the calibration curve (not shown) that measurements up to a concentration of approximately 1200 ppm is still possible, but any higher concentrations cannot be measured with this formulation.

TABLE 9
Measurement repeatability (PH) and
accuracy of SM-MB-7055 biocide.

| Accep- | Calcu- | Peak height (PH) | | | | Accuracy (%) |
|---|---|---|---|---|---|---|
| tance | lated | mean, | | RSD | Measured | Measured |
| tests | ppm | n = 3 | stdev | (%) | ppm | vs calc. |
| 2-point | 0 | 492.7 | 2.5 | 0.5% | | |
| cal. | 625 | 203.3 | 2.1 | 1.0% | | |
| Low | 100 | 375.7 | 2.9 | 0.8% | 102.2 | 2.2 |
| Medium | 1000 | 132.0 | 2.6 | 2.0% | 971.2 | −2.9 |

### Slug Detection Mode of Biocides

A biocide batch treatment is most common in oil and gas industry for various reasons. It is generally more cost-effective than continuous treatment in large oilfield systems over a long period of time. If applied properly, it can achieve the same goal as the continuous treatment—that is to bring microbial activities or counts under control in the target systems. In batch treatment, the biocide (250-1500 ppm) is injected into the target system at a pre-determined frequency (weekly being the most common) and for a pre-determined period of time (1-4 hours being the most common). In some situations, two biocides with different chemistries are used, alternating on weekly basis, for the purpose of avoiding or delaying the development of bacterial resistance or selection against the biocides.

To reduce the consumption of the reagents, and thereby also reduce the reagent refilling frequency, slug/non-slug detection mode was implemented in the GLUT analyzer, in accordance with one or more embodiments. When the system water contains no biocide slug, the analyzer conducts the measurement cycles at longer intervals to save the reagents; however, when the analyzer detects the arrival of the biocide slug, it is configured to automatically switch to slug detection mode to measure the biocide residuals as many times as possible before the biocide slug passes the analyzer.

The determination between slug and non-slug measurements was implemented in the analyzer by means of a comparator function on the measured concentration of the product in ppm. For instance, the comparator function can be set at 80 ppm±20% (i.e. ±16 ppm) of the product (see, FIG. 6), which is called “plug cut-off concentration and hysteresis.” After each measurement the log output from the control software was closely monitored by the operator and the determination that were made by the analyzer were assessed for the following situations: 1) system in non-slug mode: stay in non-slug mode, if the measured concentration<64 ppm; 2) system in non-slug mode: go to slug mode, if the measured concentration>96 ppm; 3) system in slug mode: stay in slug mode, if the measured concentration>96 ppm; and 4) system in slug mode: go to non-slug mode, if the measured concentration<64 ppm.

Slug simulation was performed as part of acceptance tests (Table 10, below). While running the GLUT analyzer with blank seawater (SW), a sample of biocide product (BT1451) was added to the blank SW volume, aiming at an approximate product concentration of >96 ppm. Following the measurement of the spiked seawater (i.e., with BT1451), the sample supply was reverted to blank seawater and the next measurement was performed. During these alternations of biocide addition and removal, the system was monitored to determine whether the GLUT analyzer would switch between slug and non-slug mode automatically based on the measured biocide concentration compared to the pre-set comparator function 80±16 ppm.

The results in Table 10 indicate that the determinations of the analyzer correctly followed the concentration changes that were measured.

TABLE 10
Example of slug/non-slug measurements of biocide BT1451.

| Sample | Detected product (ppm) | Status/Change observed |
|---|---|---|
| SW/blank | <64 | non-slug |
| SW/blank | <64 | non-slug |
| Added BT1451 | >96 | non-slug −> slug |
| Added more BT1451 | >96 | slug |
| Remained on BT1451 | >96 | slug |
| SW/blank | <64 | slug −> non-slug |
| Remained on SW/blank | <64 | non-slug |
| Added BT1451 | >96 | non-slug −> slug |
| Remained on BT1451 | >96 | slug |
| SW/blank | <64 | slug −> non-slug |
| Added BT1451 | >96 | non-slug −> slug |
| Remained on BT1451 | >96 | slug |
| SW/blank | <64 | slug −> non-slug |
| SW/blank | <64 | non-slug |

### Conclusions of Acceptance Tests

The aim of the acceptance tests was to confirm functionalities of the analyzer, and determine the dynamic measurement range, measurement repeatability (RSD), and accuracy of the analyzer using commercial biocide products. The predefined criteria for the acceptance tests are <10% measurement variation (RSD) and ±20% for measurement accuracy when compared to the calculated value (Table 3). Table 11 summarizes the outcomes of acceptance tests using two GLUT-based commercial biocide products. The measurements of both GLUT-based biocides (BT1451 and SM-MB-7055) passed the predefined criteria for detection range, measurement repeatability (RSD), and accuracy.

GLUT-based SM-MB-7055 formulation also contains 5-10% Quats, which increased the background noise of the measurement signal at higher biocide concentration. The measurement of SM-MB-7055 passed the predefined criteria for the acceptance tests at up to 1000 ppm of neat product, but it was reasonably estimated that it is still possible to measure up to approximately 1200 ppm of biocide SM-MB-7055 (data not shown). 1200 ppm upper limit is sufficient for most of the biocide treatment dosage in water injection systems. Moreover, incorporating a sample dilution mechanism in the GLUT analyzer in accordance with at least one embodiment also mitigates the impact of Quats on GLUT measurement. A 2-fold dilution of the sample is sufficient to measure up to 2000 ppm of SM-MB-7055 biocide (or 400 ppm GLUT).

During the acceptance tests, it was concluded that the comparator logic performed as expected, i.e., the system switched from non-slug mode to slug mode and vice versa upon crossing the threshold of 80 ppm±20% biocide concentration.

TABLE 11
Summary of acceptance tests of GLUT analyzer for biocide products.

| | | | | Accuracy | |
|---|---|---|---|---|---|
| | | | | (%) | |
| | | Detection range | Repeatability | measured vs | |
| | | (ppm) | (RSD) | calc. | Acceptance |
| Biocide | Criteria | 80-2000 | <10% | ±20% | tests |
| BT1451 | Calibration | 0-4000 | 0.3-2.3% | — | Pass |
| | curve | | | | |
| | Validation at | 100/1000/2000 | 0.7-3.2% | −13.7~5.4% | |
| | L/M/H* | | | | |
| SM-MB- | Calibration | 0-1200 | 1.2-5.6% | — | Pass† |
| 7055 | curve | | | | |
| | Validation at | 100/1000 | 0.8-2.0% | −2.9~2.2% | |
| | L/M | | | | |

*L/M/H: low-medium-high concentration of biocide product.
†Pass at ~1200 ppm of SM-MB-7055.

### Field Demonstration of GLUT Analyzer

A field demonstration was conducted at a large seawater injection pipeline network. The analyzer was connected to the sampling port of a seawater shipping line at a water supply plant, 93 km downstream of the biocide injection site, where BIOC31705A (THPS-based) and BIOC31450A (GLUT-based) biocides were injected alternatively on weekly basis at 750 ppm for 90 minutes. The main ingredients for these two biocides are shown in Table 12 below. For the field demonstration, the GLUT analyzer was used for online detection and quantification of the GLUT-based BIOC31450A residual when it arrives at the sampling port of the supply plant.

TABLE 12
The biocide products used in SW pipeline network.

| | | | Ethylene | |
|---|---|---|---|---|
| Biocide name | THPS | GLUT | alcohol | Methanol |
| BIOC31705A | 30-60% | | | |
| BIOC31450A | | 10-30% | 10-30% | 1-10% |

### GLUT Analyzer Installation

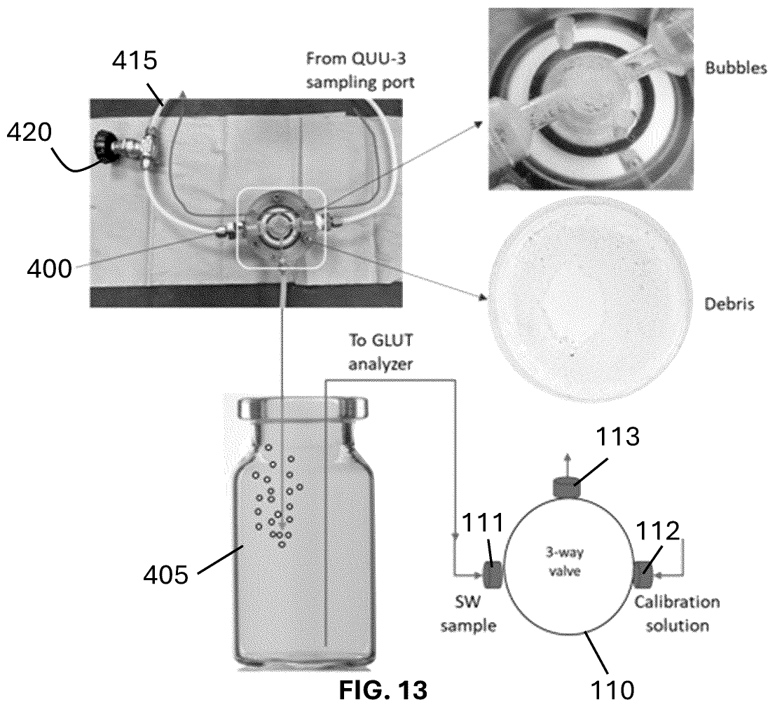

The GLUT analyzer was installed in a field laboratory at the supply plant, 93 km downstream of the biocide injection site. It was connected to the sampling port of a 60″ seawater shipping line for automated detection and monitoring of biocide slug when it arrived at the supply plant. The connection the seawater sampling port and the analyzer was configured as shown in FIG. 13 to address some challenges faced during installation and demonstration.

Two biocides (based on THPS or GLUT) were injected at the seawater treatment plant weekly and on alternating basis, meaning the THPS-based biocide would arrive at the supply plant every two weeks. The planned injection dosage was 750 ppm for 90 minutes.

The seawater shipping line is a pressurized system, and the seawater contains dissolved nitrogen gas used in a deaeration process. When the pressure is reduced from approximately 8 bar at the sampling port to a few millibars at the prototype GLUT analyzer, a large amount of gas (bubbles) is released from the seawater stream, which interferes the measurement of fluorescence signals. In addition, the treated seawater contains 0.2 mg/L or more of total suspended solids (TSS), which can clog the analyzer's flow system or interfere with the fluorescence measurement.

FIG. 13 displays an exemplary connection of the GLUT analyzer and a water sampling port (e.g., seawater sampling port) connected to a water system in accordance with one or more embodiments. FIG. 13 displays several additional features, one or more of which can be implemented as a part of the GLUT analyzer of the present application and its connection to a water system in accordance with one or more embodiments. As exemplified in FIG. 13, the solid and bubble issues discussed above were addressed by installing a filter 400 device up-side-down, an intermediate small vial 405, and a de-bubbler (not shown). More specifically, the incoming seawater goes through the filter device 400 to remove debris and most of the bubbles in the seawater line. The filters used in the filter device 400 used were GF/A Glass Microfiber Filter (Whatman Cat. No. 1820 047, 1.6 μm pore size, 47 mm diameter) and Ashless Grade 42 Quantitative Filter Paper (Whatman Cat. No. 1442 047, 2.5 μm pore size, 47 mm diameter), although it should be understood that other types of filters can be used in other embodiments.

The filter device 400 is placed in a way (i.e., upside-down) that the bubbles will stay above the filter and the majority of the seawater (along with bubbles) bypasses the filter and directly flows back to the drain 415 of the seawater sampling port. The seawater sample line from the bottom outlet of the filter device 400 goes to the small intermediate sample vial 405, from where a sample line is connected to the GLUT analyzer via the 3-way valve 110. The setup of intermediate sample vial 405 further removes the bubbles in the seawater sample. A debubbler (480 μl capacity, −0.99 barg vacuum pump) was also installed before the reaction coil for removing the sporadic bubbles in the seawater stream.

With continued reference to FIG. 13, the flow rate of the seawater flowing through the GLUT analyzer is controlled by a valve 420 in the main seawater line located behind the filter device 400. Closing this valve 420 pushes more seawater flowing through the analyzer. In the flow system of this experiment, a flow rate between 140 μl/min and 150 μl/min was desired, which was challenging to achieve with the valve 420 in the main seawater line, without creating a backpressure on the sample intake port of the 6-way valve (not shown). As such, the intermediate sample vial 405 was used allowing sampling at atmospheric pressure, which eliminated the backpressure to the sample intake port of the 6-way valve, as well as further removing bubbles in the flow system.

The GLUT analyzer was connected to the seawater shipping line with total dissolved solids (TDS) of seawater at 55˜57 g/L. Salt precipitation from the seawater can clog almost all parts of the flow system if dried out. The Au-BSA reagent solution is a sticky solution, and can build up in the tubing, clog the tubing, and thus reduce the flow rate-especially in the reaction coil, which has a very small diameter. The bending area of the reaction coil, which creates a turbulent flow for the mixing of the sample and reagent, is prone to buildup of reaction product or precipitates from the seawater, causing clogging of flow system or reduced flow rate. The clogging issue was mitigated by proper maintenance and manual de-clogging procedures as needed. The maintenance includes washing the flow system with 1% of 20% triton-100 (or Tween-20) solution before the analyzer start-up and shutdown, followed by washing with distilled water.

In this implementation, measuring the sample flow rate (140˜150 μl/min) and reagent injection rate (50 μl/stroke) was necessary before the analyzer start-up in order to determine if the analyzer had a clogging issue in the flow system. To manually de-clog, a syringe filled with triton or distilled water was used to remove the clogging materials in the flow system. If necessary, segment-to-segment de-clogging of the flow system is performed to pinpoint the exact clogging location.

The seawater pipeline was treated with two biocide products at the treatment plant, 93 km upstream of the installed GLUT analyzer (see Table 12, above). The GLUT-based biocide was planned to be injected every 2 weeks at 750 ppm for 90 minutes. However, there were deviations from the plan. For instance, some injections were delayed or cancelled due to operation issues or long holiday. In addition, the biocide treatment operation was challenged to maintain the planned injection dosage and time with outdated equipment and control system.

The GLUT analyzer was installed in a field laboratory at the water supply plant and connected to the seawater shipping line for 26 weeks for the field demonstration. The GLUT analyzer automatically measures BIOC31450A biocide residual concentrations when the biocide slug arrives at this location. From week 8 to 26, there were 6 biocide injections and 4 cancelled injections due to operation issues or long holiday (Table 13, below). Biocide slugs were detected at the supply plant were automatically detected by the GLUT analyzer at all 6 biocide injections.

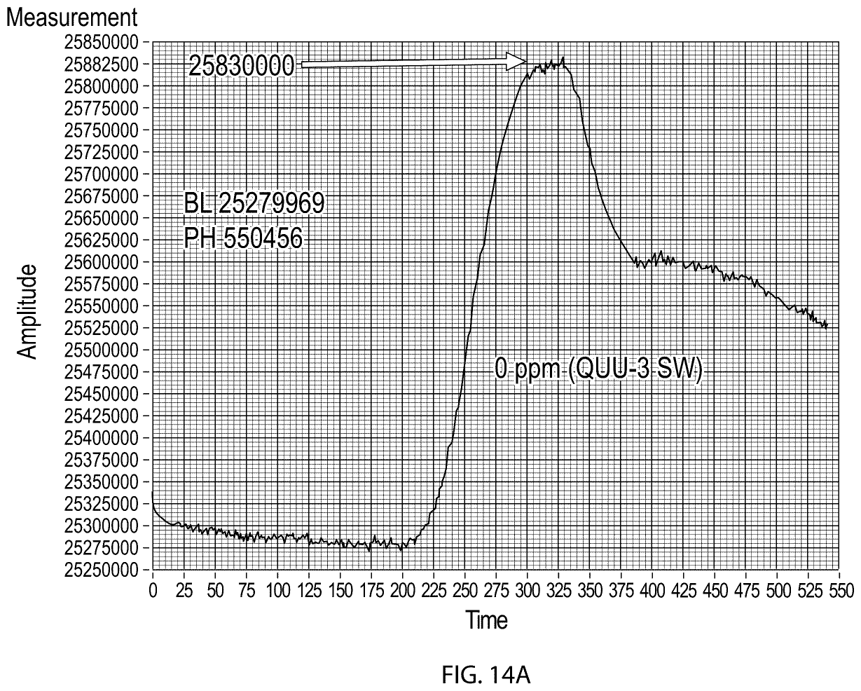

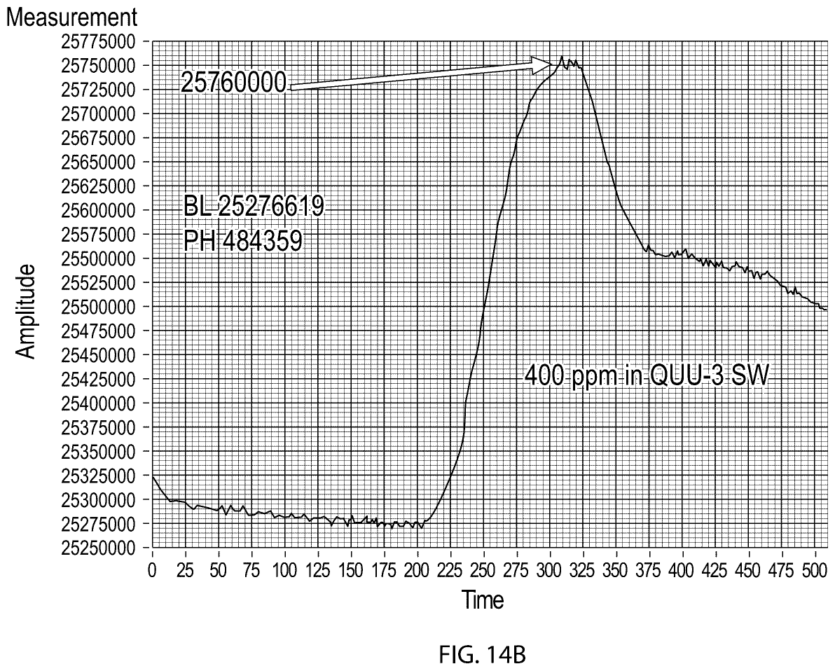

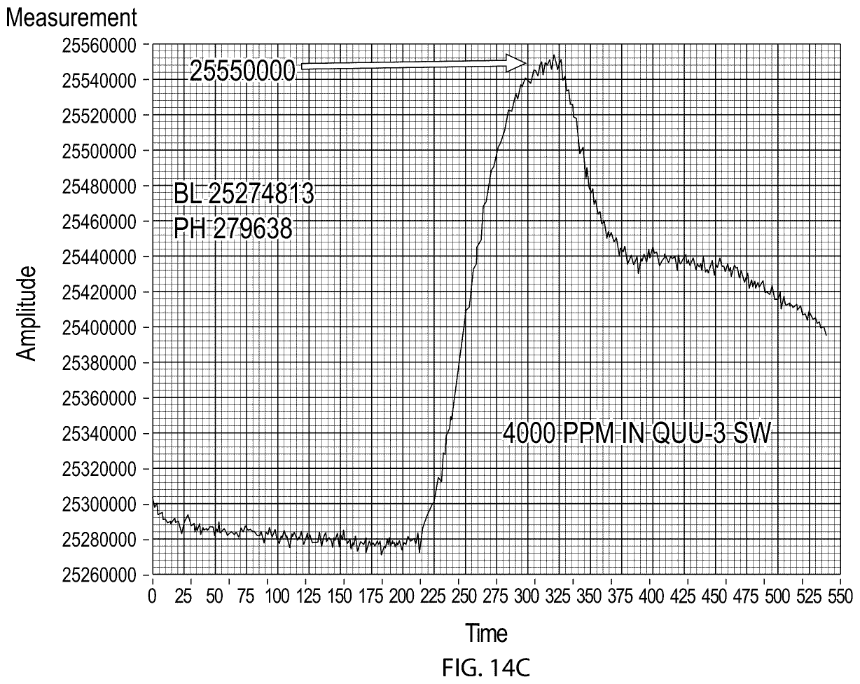

Three exemplary measurement curves for various concentrations of GLUT biocide (BIOC31450A) are illustrated in FIGS. 14A-14C. Fluorescence peak height (PH) is the value of the highest signal at the peak minus the baseline (BL) signal. As exemplified in FIGS. 14A-14C, the value of the PH is inversely proportional to the concentration of GLUT biocide in the sample, i.e., the lower the concentration, the higher the peak.

TABLE 13
GLUT biocide slug detection during field demonstration at a field laboratory.

| | | Injection | | | |
|---|---|---|---|---|---|
| Week | Date | time | ETA at Supply Plant | | Remarks |
| 1-6 | Feb. 6, 2022 | 10:00 AM | Feb. 7, 2023 | 5:37 AM | Installation, operation, |
| | Feb. 20, 2022 | 10:20 AM | Feb. 21, 2023 | 5:45 AM | trouble-shooting and |
| | Mar. 7, 2023 | 1:00 AM | Mar. 7, 2023 | 8:25 PM | calibration |
| | Mar. 20, 2023 | 10:10 AM | Mar. 21, 2023 | 5:35 AM | |
| 8 | Apr. 6, 2023 | 9:55 PM | Apr. 7, 2023 | 3:39 PM | slug detected |
| 10 | Apr. 18, 2023 | 12:10 AM | Apr. 18, 2023 | 11:06 PM | slug detected |
| 12 | May 8, 2023 | 9:00 AM | May 9, 2023 | 7:07 AM | slug detected |
| 14 | May 25, 2023 | 10:00 AM | May 26, 2023 | — | Injection cancelled |
| 16 | Jun. 5, 2023 | 9:00 AM | Jun. 6, 2023 | 4:25 AM | slug detected |
| 18 | Jun. 26, 2023 | 9:00 AM | Jun. 27, 2023 | — | Long holiday. No injection |
| | | | | | and measurement |
| 19 | Jul. 3, 2023 | 9:00 AM | Jul. 4, 2023 | — | QUU-3 pipeline isolated. No |
| 22 | Jul. 18, 2023 | 3:00 PM | Jul. 19, 2023 | — | injection. |
| 24 | Jul. 31, 2023 | 8:00 AM | Aug. 1, 2023 | 8:07 AM | slug detected |
| 26 | Aug. 14, 2023 | 9:00 AM | Aug. 15, 2023 | 9:22 AM | slug detected |

A biocide injection and the automated monitoring of biocide residual concentration at the water supple plant by the GLUT analyzer are elaborated on below as an example.

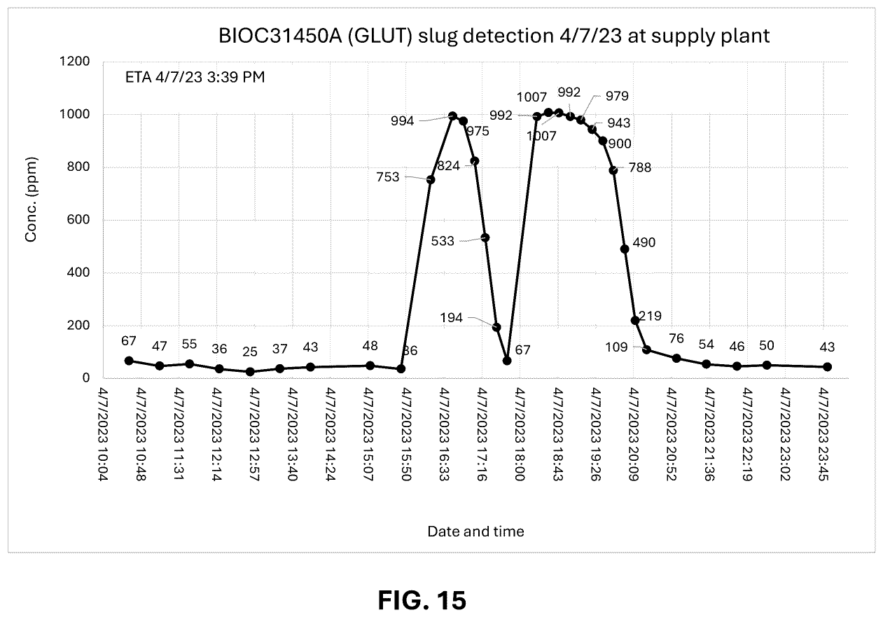

A GLUT-based BIOC31450A biocide was injected at the injection site of the treatment plant on Apr. 6, 2023, and the estimated time of arrival at the water supply plant was 3:39 μm, Apr. 7, 2023. The actual arrive time of the slug was around 4:15 μm, and the slug passed the location at around 8:10 μm, with the highest residual concentration detected at 1000 ppm (see Table 14 and FIG. 15). The results indicated that the biocide was injected at a higher concentration than the planned dosage (750 ppm) at the seawater treatment plant (93 km upstream), and the injection duration was also longer than planned (75 minutes). The GLUT analyzer was calibrated at 0 ppm and 625 ppm of GLUT biocide (2-point calibration) in the filtered seawater.

TABLE 14
GLUT biocide slug measurement on 4/7/2023 at the Supply Plant.

| Date and time | ppm | BL | PH |
|---|---|---|---|
| Apr. 7, 2023 10:34 | 67 | 25226795 | 366293 |
| Apr. 7, 2023 11:09 | 47 | 25224305 | 386353 |
| Apr. 7, 2023 11:43 | 55 | 25228818 | 378072 |
| Apr. 7, 2023 12:17 | 36 | 25221323 | 399736 |
| Apr. 7, 2023 12:52 | 25 | 25227950 | 415135 |
| Apr. 7, 2023 13:26 | 37 | 25228959 | 398800 |
| Apr. 7, 2023 14:01 | 43 | 25229003 | 391505 |
| Apr. 7, 2023 15:09 | 48 | 25230804 | 385163 |
| Apr. 7, 2023 15:44 | 36 | 25228111 | 399728 |
| Apr. 7, 2023 16:18 | 753 | 25227128 | 70367 |
| Apr. 7, 2023 16:43 | 994 | 25234767 | 7478 |
| Apr. 7, 2023 16:55 | 975 | 25236319 | 12166 |
| Apr. 7, 2023 17:08 | 824 | 25233680 | 51011 |
| Apr. 7, 2023 17:20 | 533 | 25234345 | 137160 |
| Apr. 7, 2023 17:33 | 194 | 25233336 | 278227 |
| Apr. 7, 2023 17:45 | 67 | 25235945 | 366576 |
| Apr. 7, 2023 18:19 | 992 | 25231921 | 7980 |
| Apr. 7, 2023 18:32 | 1007 | 25233154 | 4313 |
| Apr. 7, 2023 18:44 | 1007 | 25232891 | 4530 |
| Apr. 7, 2023 18:57 | 992 | 25231603 | 7985 |
| Apr. 7, 2023 19:09 | 979 | 25230027 | 11281 |
| Apr. 7, 2023 19:22 | 943 | 25229933 | 20151 |
| Apr. 7, 2023 19:34 | 900 | 25232560 | 30948 |
| Apr. 7, 2023 19:46 | 788 | 25233579 | 60618 |
| Apr. 7, 2023 19:59 | 490 | 25232967 | 151965 |
| Apr. 7, 2023 20:11 | 219 | 25232150 | 264544 |
| Apr. 7, 2023 20:24 | 109 | 25232117 | 331884 |
| Apr. 7, 2023 20:58 | 76 | 25230437 | 357970 |
| Apr. 7, 2023 21:32 | 54 | 25228726 | 379065 |
| Apr. 7, 2023 22:07 | 46 | 25229375 | 388021 |
| Apr. 7, 2023 22:41 | 50 | 25229251 | 383270 |
| Apr. 7, 2023 23:50 | 43 | 25227766 | 390708 |

### Summary of Field Demonstration

Although many deviations from the plan were encountered during the field demonstration, in terms of injection interval, dosage, and duration, due to operation issues, outdated equipment and control system, the GLUT analyzer successfully detected every biocide slug at the supply plant at the time around the estimated time of arrival.

GLUT Analyzer with Additional Features

In one or more embodiments, additional features can be added to the GLUT analyzer—and in particular the aspects of the GLUT analyzer that handles the liquid components—in order to further minimize certain technical deficiencies that can arise when the analyzer is implemented in certain environments. These deficiencies can include the need for additional bubble removal, flow rate (FR) fluctuations, backpressure, mixing reactor clogging, and varied mixing of the water sample and the reagent, for example.

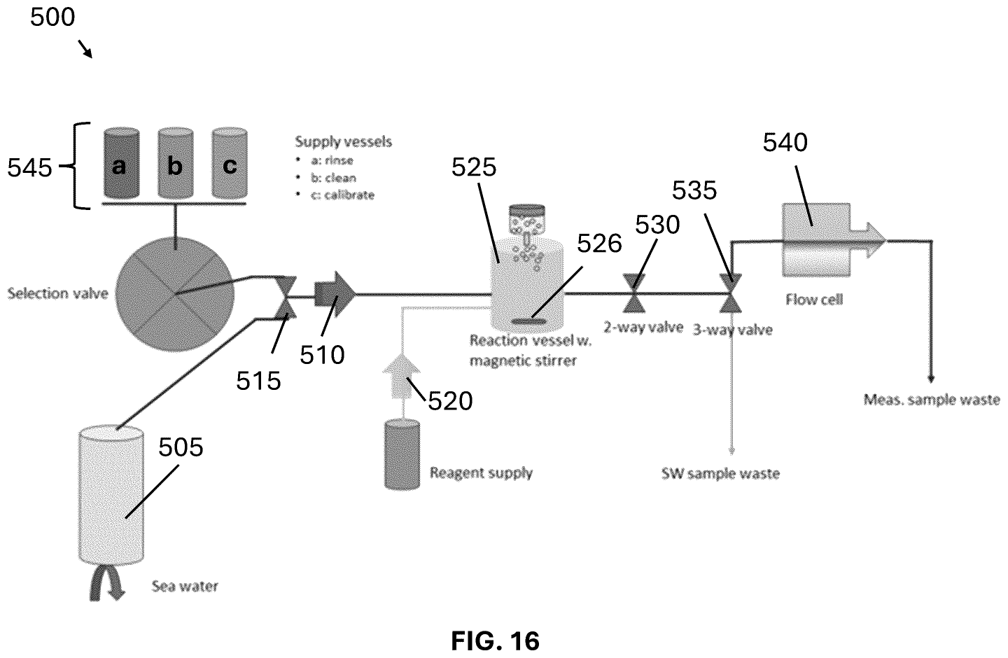

More specifically, in certain implementations large amount of bubbles can develop in the seawater line, which interfere with the measurement of absorption signals. Likewise, in certain implementations, backpressure can develop in the water sample line, causing the tubing carrying the water sample to pop out, leak, and decrease the flow rate of the sample, leading to quick wear-and-tear of the tubing. Regarding flow rate stability, in certain implementations, flow rate fluctuations can develop due to one or more factors, such as bubbles, clogging, backpressure, and tubing wear-and-tear. The peristaltic pump tubing can also require frequent replacement in certain implementations. Finally, in certain implementations, the reaction coil (e.g., a knitted coil) can become clogged, especially at a bending corner. The coil can become prone to solids buildup, causing flow rate decrease, and routine cleaning maintenance with triton and distilled water is not always sufficient to prevent or remove the clogging. In view of these issues that can arise, in certain implementations, a GLUT analyzer that includes one or more additional features as exemplified in FIG. 16 is provided.

Specifically, FIG. 16 displays a GLUT analyzer 500 comprising additional features for the liquid handling aspects of the analyzer in accordance with one or more embodiments. In one or more embodiments, the GLUT analyzer 500 can be implemented in a similar manner as the analyzer of FIGS. 1A-1C and 4A-4C, including being implemented in one or more cabinets, for example. With reference to FIG. 16, the GLUT analyzer 500 can include a cross-filter 505 which is used to remove debris and most of bubbles. The cross filter 505 can be operatively connected to the water (e.g., seawater) that the water sample is collected from. Additionally, a piston pump 510 can be included to move the water sample continuously via a 2/3-way valve 515 through to the reaction vessel 525 and the rest of the analyzer 500. It should be understood that during a calibration cycle, the piston pump 510 moves the calibration solution same way as it moves the water sample through the analyzer. In one or more embodiments a metering pump 520 can also be included, where the Au-BSA nanocluster reagent solution is injected via the metering pump 520 to a reaction vessel 525. The reaction vessel 525 in this embodiment replaces the reaction coil in the embodiments shown in FIGS. 1A-1C in implementations in which the reaction coil is prone to clogging, which can impact flow stability and measurement accuracy. In certain implementations, the reaction vessel provides 1) a consistent mixing of GLUT from the water sample or calibration solution and the Au-BSA nanocluster reagent solution, 2) stable flow, 3) sampling at atmospheric pressure, and 4) bubble removal, thereby improving the measurement accuracy.

The water sample or calibration solution is mixed with the reagent at a reaction vessel, where the bubbles are further released. The reaction vessel 525, in certain embodiments, can include a magnetic stirrer 526 for hastening the reaction between the GLUT in the water sample and the reagent. A downstream 2-way valve 530 can be closed while the water sample and the reagent are mixed in the reaction vessel.

After mixing, the mixed solution of the water sample and the reagent moves through the 2-way valve 530 (now opened) and the 3-way valve 535 to the detector 540 comprising the fluorescence flow cell for fluorescence measurement of Au-BSA nanoclusters, which is correlated with the concentration of GLUT in the water sample or calibration solution. The fluorescence flow cell of the detector 540 operates in the same fashion as the fluorescence flow cell described above with reference to FIGS. 1A-1C.

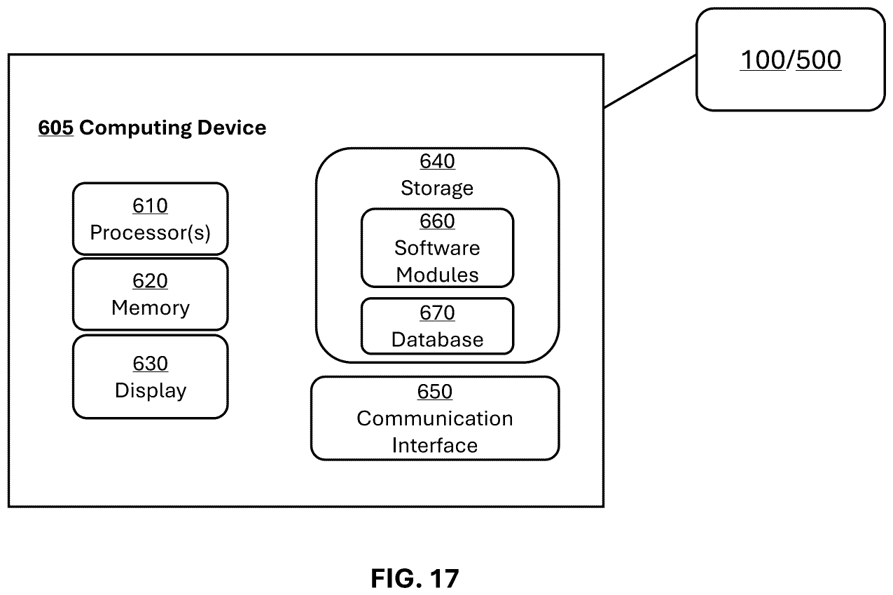

In the embodiment of the analyzer 500 in FIG. 17, the upside-down filter setup, intermediate sample vial, and de-bubbler as described in the embodiment of FIG. 13 are not included. These components were designed to remove debris and eliminate the large amount of the bubbles in the de-pressured water line at atmospheric sampling pressure. However, in the embodiment of the analyzer 500 of FIG. 17, the cross-flow filter 505 and the reaction vessel 525 with magnetic stirrer 526 achieve the same desired functionalities, and in certain implementations, can provide additional benefits.

Similarly, the piston pump 510 replaces the peristaltic pump and tubing as shown in the embodiments shown in FIGS. 1A-1C. In certain embodiments, the piston pump 510 can further improve the ruggedness and resilience of the analyzer.

Additionally, in one or more embodiments, the analyzer 500 can include an automated cleaning and rinsing feature 545 to further mitigate any clogging issues caused by solid buildup in the liquid handling aspects of the analyzer. The automated cleaning and rinsing cycles ease the required maintenance for the industrial analyzer.

Other modifications can be implemented across the various embodiments of the GLUT analyzer of the present application. For example, the temperature of seawater in the shipping line varies widely daily and seasonally, which can affect baseline signals in different seasons, and therefore affects the calibration and measurement accuracy. Accordingly, in one or more embodiments, the analyzer includes an integrated cooling and heating mechanism to maintain a stable sample and analyzer cabinet temperature. The stable cabinet temperature can also improve the stability of optical and electronic systems.

As discussed above, in one or more embodiments, the GLUT analyzer of the present application can be operatively connected to a data processor. More specifically, in one or more embodiments the GLUT analyzer disclosed herein can be part of or integrated with a computer implemented system, which can be configured with one or more data processors, memory, a controller, and a display, for example to process data received from connected devices into visual information.

FIG. 17 is a block diagram illustrating an exemplary configuration of an exemplary computing device 605 of the system for determining the presence and concentration of GLUT in a water sample, which can be operatively connected to a GLUT analyzer 100/500 of the present application in accordance with one or more embodiments. The computing device 605 includes various hardware and software components that serve to enable operation of the system, including one or more data processors 610, a memory 620, a display 630, a storage 640 and a communication interface 650. Processor 610 serves to execute software instructions that can be loaded into memory 620. Processor 610 can be a number of processors, a multi-processor core, or some other type of processor, depending on the particular implementation.

Preferably, memory 620 and/or storage 640 are accessible by processor 610, thereby enabling the processor 610 to receive and execute instructions stored on memory and/or on storage. Memory can be, for example, a random access memory (RAM) or any other suitable volatile or non-volatile computer readable storage medium. In addition, memory can be fixed or removable. Storage 640 can take various forms, depending on the particular implementation. For example, storage can contain one or more components or devices such as a hard drive, a flash memory, a rewritable optical disk, a rewritable magnetic tape, or some combination of the above. Storage also can be fixed or removable.

One or more software modules, generally indicated at 660, can be encoded in storage 640 and/or in memory 620. The software modules can comprise one or more software programs or applications having computer program code or a set of instructions executed in processor 610. Such computer program code or instructions for carrying out operations for aspects of the systems and methods disclosed herein and can be written in any combination of one or more programming languages. The program code can execute entirely on the computer device 605, as a stand-alone software package, partly on computer device 605, or entirely on another computing/device or partly on another remote computing/device. In the latter scenario, the remote computing device can be connected to computer device 605 through any type of direct electronic connection or network, including a local area network (LAN) or a wide area network (WAN), or the connection can be made to an external computer (for example, through the Internet using an Internet Service Provider).

For example, included among the software modules can be a calibration module that receives and processes the data from a 2-point calibration procedure (e.g., calibration factors a and b) which can then be stored in memory for used during measurements of water samples with unknown amounts of GLUT. Other modules can be provided to perform the functions described herein, such as temperature control of the heater; processing of each water sample, including the drawing in of each water sample and the disposal of each sample as waste after measurements have been conducted; a user interface module; etc. that are executed by processor 610. During execution of the software modules, the processor 610 configures the computer device 605 to perform various operations relating to the GLUT analyzer.

It can also be said that the program code of software modules and one or more computer readable storage devices (such as memory and/or storage) form a computer program product that can be manufactured and/or distributed in accordance with the present invention, as is known to those of ordinary skill in the art.

It should be understood that in some illustrative embodiments, one or more of software modules can be downloaded over a network to storage from another device or system via communication interface for use within the system. In addition, it should be noted that other information and/or data relevant to the operation of the present systems and methods (such as database) can also be stored on storage, as will be discussed in greater detail below.

Also preferably stored on storage is database 670. The database contains and/or maintains various data items and elements that are utilized throughout the various operations of the system. The information stored in database can include but is not limited to, parameter adjustment algorithms, set-points, settings, alarms, actual values for process variables, and historical data collected and analyzed by the computer device 605. It should be noted that although database 670 is depicted as being configured locally to computer device 605 in certain implementations database and/or various of the data elements stored therein can be located remotely (such as on a remote computing device or server—not shown) and connected to computer device 605 through a network or in a manner known to those of ordinary skill in the art.

The communication interface 650 is also operatively connected to the processor 610. The interface can be one or more input device(s) such as switch(es), button(s), key(s), a touch-screen, microphone, etc. as would be understood in the art of electronic computing devices. The interface 650 serves to facilitate the capture of commands from the user such as on-off commands or settings related to operation of the analyzer.

The display 630 is also operatively connected to processor 610. Display 630 can include a screen or any other such presentation device which enables the user to view information relating to operation of the system including control settings, command prompts and data collected by various components of the system and provided to computer device 605. By way of further example, interface 650 and display 630 can be integrated into a touch screen display. Accordingly, the screen is used to show a graphical user interface, which can display various data and provide “forms” that include fields that allow for the entry of information by the user. Touching the touch screen at locations corresponding to the display of a graphical user interface allows the person to interact with the device to enter data, change settings, control functions, etc. So, when the touch screen is touched, interface 50 communicates this change to processor 610, and settings can be changed or user entered information can be captured and stored in the memory.

Communication interface 650 is operatively connected to the processor 610 and can be any interface that enables communication between the computer device 605 and external devices, machines and/or elements. Preferably, communication interface 650 includes, but is not limited to, Ethernet, IEEE 1394, parallel, PS/2, Serial, USB, VGA, DVI, SCSI, HDMI, a Network Interface Card (NIC), an integrated network interface, a radio frequency transmitter/receiver (e.g., Bluetooth, cellular, NFC), a satellite communication transmitter/receiver, an infrared port, and/or any other such interfaces for connecting computer device 605 to other computing devices and/or communication networks such as private networks and the Internet. Such connections can include a wired connection (e.g., using the RS232 standard) or a wireless connection (e.g., using the 802.11 standard) though it should be understood that communication interface can be practically any interface that enables communication to/from the computer device 605.

The processor(s) 610 can be configured, for example, by executing instructions stored on non-transitory processor readable media, to process information of various types and from various sources, including live visual data and data provided via one or more of sensors, instrument controllers, and display(s).

In one or more embodiments, the one or more processors can be configured by executing instructions, for example, provided in a series of software and/or hardware modules, to interpret, manipulate and record information received from the site at which each water sample is capture. Moreover, the processors can be configured to manipulate and provide illustrative and graphical overlays, and generate composite or hybrid visual data to the display device. The analyzer system can further include haptic technology that provides vibratory or other feedback in response to information processed by one or more processors.

Although much of the foregoing description has been directed to, the apparatus and methods disclosed herein can be similarly deployed and/or implemented in scenarios, situations, and settings far beyond the referenced scenarios. It should be further understood that any such implementation and/or deployment is within the scope of the methods described herein.

It is to be further understood that like numerals in the drawings represent like elements through the several figures, and that not all components and/or steps described and illustrated with reference to the figures are required for all embodiments or arrangements. Further, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “including,” “comprising,” or “having,” “containing,” “involving,” and variations thereof herein, when used in this specification, specify the presence of stated features, integers, steps, operations, elements, and/or components, but do not preclude the presence or addition of one or more other features, integers, steps, operations, elements, components, and/or groups thereof.

It should be noted that use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Notably, the figures and examples above are not meant to limit the scope of the present disclosure to a single implementation, as other implementations are possible by way of interchange of some or all the described or illustrated elements. Moreover, where certain elements of the present disclosure can be partially or fully implemented using known components, only those portions of such known components that are necessary for an understanding of the present disclosure are described, and detailed descriptions of other portions of such known components are omitted so as not to obscure the disclosure. In the present specification, an implementation showing a singular component should not necessarily be limited to other implementations including a plurality of the same component, and vice-versa, unless explicitly stated otherwise herein. Moreover, applicants do not intend for any term in the specification or claims to be ascribed an uncommon or special meaning unless explicitly set forth as such. Further, the present disclosure encompasses present and future known equivalents to the known components referred to herein byway of illustration.

The foregoing description of the specific implementations will so fully reveal the general nature of the disclosure that others can, by applying knowledge within the skill of the relevant art(s), readily modify and/or adapt for various applications such specific implementations, without undue experimentation, without departing from the general concept of the present disclosure. Such adaptations and modifications are therefore intended to be within the meaning and range of equivalents of the disclosed implementations, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s). It is to be understood that dimensions discussed or shown are drawings are shown accordingly to one example and other dimensions can be used without departing from the disclosure.

The subject matter described above is provided by way of illustration only and should not be construed as limiting. Various modifications and changes can be made to the subject matter described herein without following the example embodiments and applications illustrated and described, and without departing from the true spirit and scope of the invention encompassed by the present disclosure, which is defined by the set of recitations in the following claims and by structures and functions or steps which are equivalent to these recitations.

What is claimed is:

1. An analyzer for detecting and quantifying a glutaraldehyde (GLUT) composition in a water sample, the analyzer comprising:
a peristaltic pump and a three-way valve operatively connected to the peristaltic pump, wherein the peristaltic pump is configured to draw in a water sample comprising a GLUT composition through the 3-way valve;
a six-way valve operatively connected to the three-way valve via the peristaltic pump, wherein the six-way valve is configured to receive: a) the water sample from the 3-way valve via a first inlet and b) a bovine serum albumin-coated gold (Au-BSA) nanoclusters reagent via a second inlet;
a debubbler operatively connected to the six-way valve and configured to receive a mixture of the water sample and the Au-BSA nanoclusters reagent and remove bubbles from the mixture; and
a reaction coil operatively connected to the debubbler and configured to receive the water sample and the Au-BSA nanoclusters reagent, wherein the water sample and the Au-BSA nanoclusters reagent are mixed and reacted in the reaction coil;
a fluorescence flow cell comprising a 375 nm light-emitting diode (LED), wherein the flow cell is configured to:
receive the mixture from the reaction coil;
measure a fluorescence intensity of the Au-BSA nanoclusters in the mixture at an emission wavelength of 675 nm, wherein the Au-BSA nanoclusters comprises a maximum excitation wavelength of approximately 375 nm and a maximum emission wavelength of approximately 675 nm; and
determine a presence and concentration of the GLUT composition in the water sample based on a comparison between the measured fluorescence intensity of the Au-BSA nanoclusters at the emission wavelength of 675 nm with fluorescence intensity values of calibration samples comprising Au-BSA nanoclusters and known glutaraldehyde concentrations.

2. The analyzer of claim 1, wherein a correlation of the fluorescence intensity of the Au-BSA nanoclusters and the concentration of the GLUT composition is based on the equation: Y=a+b\sqrt{X}, wherein Y is the peak height of the fluorescence signal, X is the concentration of GLUT composition in the water sample, and a and b are calibration factors determined using a 2-point calibration procedure.

3. The analyzer of claim 2, wherein to perform the 2-point calibration procedure the fluorescence flow cell is configured to:
measure a fluorescence intensity of the Au-BSA nanoclusters at an emission wavelength of 675 nm in a mixture of a water sample without the GLUT composition and the Au-BSA nanoclusters reagent to determine calibration factor a, wherein a=PH and PH is the peak height of the fluorescence signal; and
measure a fluorescence intensity of the Au-BSA nanoclusters at an emission wavelength of 675 nm in a mixture of a water sample with a known concentration c of the GLUT composition and the Au-BSA nanoclusters reagent to determine calibration factor b, wherein
$$b = \frac{P H - a}{√ c} .$$

4. The analyzer of claim 1, wherein the three-way valve comprises a water sample inlet for receiving the water sample, a calibration inlet for a calibration fluid, and an outlet configured to direct the water sample or calibration fluid to the remaining components of the analyzer.

5. The analyzer of claim 1, further comprising a reagent injector configured to inject the Au-BSA nanoclusters reagent into the 6-way valve.

6. The analyzer of claim 1, wherein the water sample is a calibration fluid, and wherein when the 3-way valve receives a calibration fluid, the analyzer is configured to perform a calibration cycle.

7. The analyzer of claim 1, wherein the analyzer has a dynamic detection range for the GLUT composition of approximately 80-2000 ppm.

8. The analyzer of claim 1, wherein the GLUT composition is a biocide composition and wherein the water is seawater.

9. The analyzer of claim 1, further comprising a heater operatively connected to the reaction coil, wherein the heater is configured to maintain the reaction coil at a temperature in the range of approximately 35-45° C.

10. The analyzer of claim 9, wherein the heater is configured to maintain the reaction coil at approximately 40° C.

11. The analyzer of claim 1, further comprising a bandpass filter configured to improve sensitivity and selectivity of the analyzer.

12. The analyzer of claim 1, wherein the measurement accuracy of the concentration of the GLUT composition is within ±20% of the actual GLUT concentration.

13. The analyzer of claim 1, wherein the analyzer is an online analyzer operatively connected to a water system and configured to perform real-time measurements of water samples from the water system.

14. The analyzer of claim 13, wherein the analyzer is configured to automatically switch from a slug mode to a non-slug mode if the measured concentration of the GLUT composition is below a first predetermined value and to switch from non-slug mode to slug mode if the measured concentration of the GLUT composition is above a second predetermined value.

15. The analyzer of claim 13, further comprising:
a filter device operatively connected to the water system via tubing that receives the water sample from the water system, wherein the filter device is configured to remove debris and bubbles from the water sample; and
an intermediate sample vial operatively connected to the three-way valve and configured to receive the water sample after passing through the filter device and further remove bubbles in the water sample,
wherein the filter device and the intermediate sample vial are located upstream of the three-way valve.

16. An online analyzer operatively connected to a water system for detecting and quantifying a GLUT composition in a water sample, the analyzer comprising:
a cross-flow filter operatively connected to the water system via tubing that receives a water sample comprising a GLUT composition from the water system, wherein the filter device is configured to remove debris and bubbles from the water sample;
a piston pump configured to draw in the water sample from the water system through the cross-flow filter;
a reaction vessel operatively connected to the piston pump and configured to receive the water sample via the piston pump and an Au-BSA nanoclusters reagent from a reagent supply, wherein the water sample and the Au-BSA nanoclusters reagent are mixed and reacted in the reaction vessel and the reaction vessel is further configured to remove bubbles from the water sample and Au-BSA nanoclusters reagent mixture;
a fluorescence flow cell comprising a 375 nm light-emitting diode (LED), wherein the flow cell is configured to:
receive the mixture from the reaction vessel;
measure a fluorescence intensity of the Au-BSA nanoclusters in the mixture at an emission wavelength of 675 nm, wherein the Au-BSA nanoclusters comprises a maximum excitation wavelength of approximately 375 nm and a maximum emission wavelength of approximately 675 nm; and
determine a presence and concentration of the GLUT composition in the water sample based on a comparison between the measured fluorescence intensity of the Au-BSA nanoclusters at the emission wavelength of 675 nm with fluorescence intensity values of calibration samples comprising Au-BSA nanoclusters and known glutaraldehyde concentrations.

17. The analyzer of claim 16, wherein a correlation of the fluorescence intensity of the Au-BSA nanoclusters and the concentration of the GLUT composition is based on the equation: Y=a+b\sqrt{X}, wherein Y is the peak height of the fluorescence signal, X is the concentration of GLUT composition in the water sample, and a and b are calibration factors determined using a 2-point calibration procedure.

18. The analyzer of claim 17, wherein to perform the 2-point calibration procedure the fluorescence flow cell is configured to:
measure a fluorescence intensity of the Au-BSA nanoclusters at an emission wavelength of 675 nm in a mixture of a water sample without the GLUT composition and the Au-BSA nanoclusters reagent to determine calibration factor a, wherein a=PH and PH is the peak height of the fluorescence signal; and
measure a fluorescence intensity of the Au-BSA nanoclusters at an emission wavelength of 675 nm in a mixture of a water sample with a known concentration c of the GLUT composition and the Au-BSA nanoclusters reagent to determine calibration factor b, wherein
$$b = \frac{P H - a}{√ c} .$$

19. The analyzer of claim 16, wherein the analyzer has a dynamic detection range for the GLUT composition of approximately 80-2000 ppm.

20. The analyzer of claim 16, wherein the measurement accuracy of the concentration of the GLUT composition is within ±20% of the actual GLUT concentration.
